(19) United States
(12) Patent Application Publication
(10) Pub. No.: US 2026/0263812 A1
(43) Pub. Date: Sep. 10, 2026

(54) SYSTEMS AND METHODS FOR PROVIDING STIMULATION

(71) Applicant: THE ALFRED E. MANN FOUNDATION FOR SCIENTIFIC RESEARCH, VALENCIA, CA (US)

(72) Inventor: Brian Shelton, San Buenaventura, CA (US)

(21) Appl. No.: 19/554,917

(22) Filed: Mar. 3, 2026

Related U.S. Application Data
(60) Provisional application No. 63/768,729, filed on Mar. 7, 2025.

(51) Int. Cl. A61N 1/36; A61N 1/05

(52) U.S. Cl. CPC A61N 1/36185; A61N 1/0556

(57) ABSTRACT

A medical system includes an implantable pulse generator (IPG); a lead electrically coupled to the IPG; and a stimulation electrode on the lead and including a plurality of separately chargeable electrode contacts, wherein the IPG includes a driver configured to apply electrical stimulus; and a first electrical switch electrically coupled to the driver and positionable in at least a first position, whereby the first electrical switch is coupled to a first electrode contact of the plurality of electrode contacts, and a second position, whereby the first electrical switch is coupled to a second electrode contact.

### CROSS-REFERENCE TO RELATED APPLICATION

This application claims priority to, and the benefit of, U.S. Provisional Patent Application No. 63/768,729, filed Mar. 7, 2025, the entire content of which is incorporated herein by reference.

### BACKGROUND

### 1. Field

The present disclosure relates to systems and methods for providing stimulation, and more particularly, to systems and methods for sequentially applying, via a driver, electrical stimulus to a plurality of electrode contacts to provide stimulation.

### 2. Description of the Related Art

An implantable stimulating device, such as a nerve cuff electrode, paddle electrode, or linear lead with an array of electrode contacts may be used to provide electrical stimulation to biological tissue, such as a nerve. It can be desirable to enable the implantable electrode to concentrate this electrical stimulation to a specific target tissue, such as a specific nerve tissue for stimulation. Targeting specific tissue by “current steering” can be done typically by controlling the flow of electrical current to the specific target tissue by selectively and differentially charging two cathode electrode contacts of the implantable stimulating device. A first electrical charge is applied to the first cathode electrode contact and a second electrical charge is applied to the second cathode electrode contact, simultaneously resulting in an overlapping current flow from the two cathodes. The flow of electrical current will be steered to a location emanating somewhere between the first and second cathode electrode contacts. To enable this mode of current steering, typically, the output stage of an implantable pulse generator (IPG) electronics of the implantable stimulating device must be configured with at least two independent current sources or drivers that can apply the first stimulus amplitude and second stimulus amplitude to the two cathode electrode contacts at the same time. However, including a plurality of separate current sources/drivers in an IPG to configure the implantable stimulating device to differentially charge a plurality of electrode contacts can disadvantageously increase the number of components in the IPG, the cost and complexity of operation of the IPG, and the size of the IPG. It is therefore desirable to provide an IPG that is configured to target specific tissue for stimulation via a single, or a limited number of, current sources/drivers.

The technology of the present disclosure is set forth in view of this technical background. This Background Section is provided only for purposes of introducing certain background material relating to the present disclosure and, thus, is not an admission of prior art.

### SUMMARY

According to an aspect, the technology relates to a medical system, including an implantable pulse generator (IPG); a lead electrically coupled to the IPG; and a stimulation electrode on the lead and including a plurality of separately chargeable electrode contacts, wherein the IPG includes a driver configured to apply electrical stimulus; and a first electrical switch electrically coupled to the driver and positionable in at least a first position, whereby the first electrical switch is coupled to a first electrode contact of the plurality of electrode contacts, and a second position, whereby the first electrical switch is coupled to a second electrode contact.

In some examples, the stimulation electrode is a nerve cuff electrode, and the plurality of electrode contacts are configured to be arranged to at least partially circumferentially surround the nerve when the nerve cuff electrode is wrapped around the nerve.

In some examples, the second electrode contact is of the plurality of electrode contacts of the stimulation electrode.

In some examples, the second electrode contact is separated from the stimulation electrode.

In some examples, the second electrode contact is on the IPG.

In some examples, the IPG includes a second electrical switch coupled to the driver and selectively positionable in each of multiple different positions, whereby the second electrical switch is respectively coupled to multiple different electrode contacts, the multiple different electrode contacts including the first and second electrode contacts, and the first electrical switch is selectively positionable in each of the multiple different positions independently of the position of the second electrical switch.

In some examples, the IPG is configured to cause the driver to be coupled, via the first and second electrical switches, to two different electrode contacts of the multiple different electrode contacts, and to apply cathodic electrical stimulus to one of the two different electrode contacts and corresponding anodic electrical stimulus to another one of the two different electrode contacts.

In some examples, the IPG further includes a microcontroller circuit operatively coupled to the driver and to the first electrical switch.

In some examples, the microcontroller circuit is configured to cause the driver to apply electrical stimulus to at least some of the plurality of electrode contacts via a time-multiplexing operation of the first electrical switch, to generate a combined electric field defined at least in part by the combination of individual electric fields respectively associated with the at least some of the plurality of electrode contacts.

In some examples, the microcontroller circuit is configured to cause the driver to differentially charge two electrode contacts of the plurality of electrode contacts with different amplitudes of an anodic charge, or with different amplitudes of a cathodic charge, to generate a combined electric field defined at least in part by the combination of individual electric fields respectively associated with the two charged electrode contacts.

In some examples, the microcontroller circuit is configured to electrically couple, via the first electrical switch, the driver to the first electrode contact and cause the driver to apply first electrical stimulus to the first electrode contact; and electrically couple, via the first electrical switch, the driver to the second electrode contact and cause, while the first electrical stimulus is applied to the first electrode contact, the driver to apply second electrical stimulus to the second electrode contact.

In some examples, the microcontroller circuit is configured to electrically couple, via the first electrical switch, the driver to a third electrode contact and cause, while the first and second electrical stimuli are respectively applied to the first and second electrode contacts, the driver to apply third electrical stimulus to the third electrode contact.

In some examples, the first electrical stimulus is cathodic electrical stimulus, and the microcontroller is configured to cause the driver to apply a third electrode contact with anodic electrical stimulus while the driver is applying the first electrode contact with the cathodic electrical stimulus.

In some examples, the third electrode contact is separated from the stimulation electrode, the second electrode contact is of the plurality of electrode contacts of the stimulation electrode, and the second electrical stimulus is cathodic electrical stimulus.

In some examples, the first electrical stimulus is anodic electrical stimulus, the second electrode contact is of the plurality of electrode contacts of the stimulation electrode, the second electrical stimulus is anodic electrical stimulus, and the microcontroller circuit is configured to cause the driver to apply cathodic electrical stimulus to a third electrode contact of the plurality of electrode contacts of the stimulation electrode while the driver is applying the first electrode contact with the anodic electrical stimulus; and apply additional cathodic electrical stimulus to the third electrode contact while the driver is applying the first electrode contact with the anodic electrical stimulus.

In some examples, the microcontroller circuit is configured to cause the first electrode contact to receive the first electrical stimulus via a first pulse provided by the driver, to hold the first electrical stimulus during a first interphase period, and to discharge the first electrical stimulus after the first interphase period; and cause the second electrode contact to receive the second electrical stimulus via a second pulse provided by the driver during the first interphase period, to hold the second electrical stimulus during a second interphase period, and to discharge the second electrical stimulus after the second interphase period.

In some examples, the microcontroller circuit is configured to cause the first electrode contact to discharge the first electrical stimulus via a plurality of first discharge pulses; and cause the second electrode contact to discharge the second electrical stimulus via a plurality of second discharge pulses.

In some examples, the plurality of second discharge pulses alternate with the plurality of first discharge pulses.

In some examples, the microcontroller circuit is configured to cause the first electrode contact to receive the first electrical stimulus via a plurality of first charge pulses provided by the driver, to hold the first electrical stimulus during a first interphase period, and to discharge the first electrical stimulus after the first interphase period; and cause the second electrode contact to receive the second electrical stimulus via a plurality of second charge pulses provided by the driver, to hold the second electrical stimulus during a second interphase period, and to discharge the second electrical stimulus after the second interphase period.

In some examples, the plurality of second charge pulses alternate with the plurality of first charge pulses.

In some examples, the microcontroller circuit is configured to cause the first electrode contact to discharge the first electrical stimulus via a plurality of first discharge pulses; and cause the second electrode contact to discharge the second electrical stimulus via a plurality of second discharge pulses that alternate with the plurality of first discharge pulses.

According to an aspect, the technology relates to a medical system, including an implantable pulse generator (IPG) configured for current steering; a lead electrically coupled to the IPG; and a stimulation electrode on the lead and including a plurality of separately chargeable electrode contacts, wherein the IPG includes a driver configured to selectively apply electrical stimulus to the plurality of electrode contacts, and wherein the IPG is configured to cause the driver to apply first electrical stimulus to a first electrode contact of the plurality of electrode contacts; and subsequently cause, while the first electrical stimulus is stored on the first electrode contact, the driver to apply second electrical stimulus to a second electrode contact.

In some examples, the IPG is configured to cause the first electrode contact to hold the first electrical stimulus during a first interphase period and to cause the driver to discharge the first electrical stimulus after the first interphase period.

In some examples, the IPG is configured to cause the driver to apply the second electrical stimulus to the second electrode contact during the first interphase period.

In some examples, the stimulation electrode is a nerve cuff electrode configured to wrap around a nerve.

In some examples, the second electrode contact is of the plurality of electrode contacts of the stimulation electrode.

In some examples, the second electrode contact is separated from the stimulation electrode.

In some examples, the second electrode contact is on the IPG.

In some examples, the IPG includes a microcontroller circuit operatively coupled to the driver.

In some examples, the first and second electrical stimuli have the same polarity, and the driver is configured to differentially apply the first and second electrical stimuli with different amplitudes.

In some examples, the IPG is configured to cause the second electrode contact to hold the second electrical stimulus during a second interphase period, and to discharge the second electrical stimulus after the second interphase period.

In some examples, the IPG is configured to cause the driver to discharge the first electrical stimulus during the second interphase period.

In some examples, the IPG is configured to cause the first and second electrode contacts to respectively discharge the first and second electrical stimuli by a plurality of discharge pulses.

In some examples, the plurality of discharge pulses for discharging the first electrode contact are alternated with the plurality of discharge pulses for discharging the second electrode contact.

In some examples, the driver is configured to apply, while applying the first electrical stimulus to the first electrode contact, a third electrode contact with third electrical stimulus of opposite polarity to the first electrical stimulus.

In some examples, the first electrical stimulus is cathodic electrical stimulus, the second electrical stimulus is cathodic electrical stimulus, the third electrical stimulus is anodic electrical stimulus, the second electrode contact is of the plurality of electrode contacts of the stimulation electrode, and the third electrode contact is separated from the stimulation electrode.

In some examples, the first electrical stimulus is anodic electrical stimulus, the second electrical stimulus is anodic electrical stimulus, the third electrical stimulus is cathodic electrical stimulus, and the second and third electrode contacts are each of the plurality of electrode contacts of the stimulation electrode, wherein the driver is configured to apply, while applying the anodic electrical stimulus to the second electrode contact, additional cathodic electrical stimulus to the third electrode contact.

According to an aspect, the technology relates to a method for stimulating a nerve, the method including providing electric current steering to the nerve by applying, via a driver, first electrical stimulus to a first electrode contact of a stimulation electrode; and subsequently applying, via the driver and while the first electrical stimulus is stored on the first electrode contact, second electrical stimulus to a second electrode contact of the stimulation electrode.

In some examples, the first and second electrical stimuli have the same polarity.

In some examples, the first and second electrical stimuli have different amplitudes.

In some examples, the first and second electrical stimuli are both cathodic electrical stimulus.

In some examples, the method further includes applying anodic electrical stimulus to a third electrode contact separated from the stimulation electrode.

In some examples, part of the anodic electrical stimulus is applied by the driver while the driver applies the cathodic electrical stimulus to the first electrode contact, and another part of the anodic electrical stimulus is applied to the third electrode contact while the driver applies the cathodic electrical stimulus to the second electrode contact.

In some examples, the third electrode contact is on an implantable pulse generator that is configured to apply electrical stimulus to the stimulation electrode through a lead.

In some examples, the first and second electrical stimuli are both anodic electrical stimulus, and the method further includes applying cathodic electrical stimulus to a third electrode contact of the stimulation electrode.

In some examples, part of the cathodic electrical stimulus is applied by the driver to the third electrode contact while the driver is applying the anodic electrical stimulus to the first electrode contact, and another part of the cathodic electrical stimulus is applied by the driver to the third electrode contact while the driver is applying the anodic electrical stimulus to the second electrode contact.

In some examples, the first and second electrical stimuli have opposite polarities.

In some examples, the first and second electrical stimuli have different amplitudes.

In some examples, the method further includes applying, via the driver and while the driver applies the first electrical stimulus to the first electrode contact, third electrical stimulus to a third electrode contact, the third electrical stimulus having opposite polarity to the first electrical stimulus.

In some examples, the method includes applying, via the driver and while the first and second electrical stimuli are stored on the first and second electrode contacts, third electrical stimulus to a third electrode contact.

In some examples, the first electrical stimulus is applied to the first electrode contact by a first stimulus pulse provided by the driver, the first electrode contact holds the first electrical stimulus during a first interphase period, and the driver discharges the first electrical stimulus from the first electrode contact after the first interphase period and by one or more second stimulus pulses of opposite polarity to the first stimulus pulse, and the second electrical stimulus is applied to the second electrode contact during the first interphase period and by a third stimulus pulse.

In some examples, the driver electrically decouples from the first electrode contact during the first interphase period.

In some examples, the second electrode contact holds the second electrical stimulus during a second interphase period, and the driver discharges the second electrical stimulus from the second electrode contact after the second interphase period and by a fourth stimulus pulse of opposite polarity to the third stimulus pulse.

In some examples, the method further includes discharging the first electrical stimulus from the first electrode contact via a plurality of second stimulus pulses, and discharging the second electrical stimulus from the second electrode contact via a plurality of third stimulus pulses.

In some examples, the plurality of second stimulus pulses are alternated with the plurality of third stimulus pulses.

In some examples, the driver includes only a single current source configured to provide current in a single direction, and the single current source is configured to be functionally bi-directional via at least one switch connected to the single current source.

In some examples, the driver includes only a single, bi-directional current source.

This Summary Section introduces some features of nonlimiting and non-exhaustive examples of the present disclosure and is not intended to limit the scope of the claims. Moreover, the present disclosure encompasses all examples of systems and methods that include any combination of features of systems and methods disclosed in this Summary Section.

### BRIEF DESCRIPTION OF THE DRAWINGS

The drawings, together with the specification, illustrate nonlimiting and non-exhaustive examples of the present disclosure.

FIG. 1 illustrates an implantable medical system (IMS), according to some examples, when implanted in a patient.

FIG. 2 illustrates the IMS of FIG. 1, according to some examples.

FIG. 3 illustrates a schematic of the IMS of FIG. 1, according to some examples.

FIG. 4 illustrates a cross-sectional schematic of a nerve and a stimulation electrode of the IMS of FIG. 1, according to some examples, stimulus waveforms applied to certain electrode contacts according to some examples, and two electric fields generated by the certain electrode contacts.

FIG. 5 illustrates FIG. 4 with the two electric fields superimposed into a combined electric field.

FIG. 6 illustrates stimulus waveforms to be applied to electrode contacts, according to some examples.

FIG. 7 illustrates stimulus waveforms to be applied to electrode contacts, according to some examples.

FIG. 8 illustrates stimulus waveforms to be applied to electrode contacts, according to some examples.

FIG. 9 illustrates stimulus waveforms to be applied to electrode contacts, according to some examples.

FIG. 10 illustrates stimulus waveforms to be applied to electrode contacts, according to some examples.

FIGS. 11A and 11B illustrate a flow chart for a process of providing electrical stimulation, according to some examples.

### DETAILED DESCRIPTION

Nonlimiting and non-exhaustive examples of systems and methods for providing stimulation will now be described in more detail with reference to the drawings. The systems described herein may include an implantable medical system (IMS) that comprises an IPG and a stimulation lead that can connect to the IPG. As an exemplary embodiment, the stimulation lead has, at the distal end, a nerve cuff electrode having a plurality of electrode contacts. For the sake of clarity, as used in this disclosure, “electrode contacts” may refer to the plurality of electrically conductive metal contacts that can function as either anodes or cathodes. An “electrode” can be, for example, the nerve cuff electrode, paddle electrode or the distal part of a linear lead. An electrode contact may also be located outside of the nerve cuff, for example on an IPG or on a second stimulation lead. The IPG can include a single driver (e.g., a single current source), or a limited number of drivers, that is configured to sequentially provide electrical stimulus to a plurality of electrode contacts, for example, via a time-multiplexing operation, in order to control the shape of an electric field and electric current generated in biological tissue near the electrode contacts. This current steering via a single driver can allow the IMS to target specific tissue (e.g., specific nerve fascicles within a nerve fiber bundle) for stimulation without requiring a large number of separate drivers, which would increase the cost, number of components, complexity of operation, and size of the IMS.

In the present disclosure, it will be understood that, although the terms “first”, “second”, “third”, etc., may be used herein to describe various elements, processes, or other features, these elements, processes, or features should not be limited by these terms. These terms are only used to distinguish one element, process, or feature from another element, process, or feature. Thus, a first element, process, or feature discussed herein could be termed a second element, process, or feature, without departing from the spirit and scope of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” specify the presence of stated elements, processes, and/or other features, but do not preclude the presence or addition of one or more other elements, processes, and/or features. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element is referred to as being “on”, “connected to”, “coupled to”, “attached to”, or “adjacent to” another element, it can be directly on, connected to, coupled to, attached to, or adjacent to the other element, or one or more intervening element(s) may be present. In contrast, when an element is referred to as being “directly on,” “directly connected to”, “directly coupled to”, “directly attached to”, or “immediately adjacent to” another element, there are no intervening elements present. Similar terms and phrases should be understood in a similar manner to encompass both direct and indirect affiliations between two or more elements being discussed. In addition, it will also be understood that when an element is referred to as being “between” two elements, it can be the only element between the two elements (e.g., it can be directly between the two elements), or one or more intervening elements may also be present.

As used herein, the phrase “at least part” includes part or all of the stated item, the phrase “at least partly” includes the stated item partly or entirely, and similar phrases should be interpreted in a similar manner.

As used herein, and unless stated otherwise, the term "substantially" and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of "1.0 to 10.0" is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The term microcontroller unit (MCU) (e.g., microcontroller circuit) is used herein to include any combination of hardware, firmware, and software, employed to process data or digital signals. Processing unit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs).

The IPG, MCU, and/or any other relevant devices or components according to embodiments of the present invention described herein may be implemented utilizing any suitable hardware, firmware (e.g. an application-specific integrated circuit), software, or a combination of software, firmware, and hardware. For example, the various components of the IPG, MCU, etc., may be formed on one integrated circuit (IC) chip or on separate IC chips. Further, the various components of the IPG, MCU, etc., may be implemented on a flexible printed circuit film, a tape carrier package (TCP), a printed circuit board (PCB), or formed on one substrate. Further, the various components of the IPG, MCU, etc., may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

Accordingly, in some examples, the functions described may be implemented in hardware, firmware, software, or any combination thereof. If implemented in firmware and/or software, the functions may be stored on or encoded as one or more instructions or code on a computer-readable medium. Computer-readable media includes computer storage media. Storage media may be any available media that can be accessed by a computer. For example, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer (e.g., by an MCU).

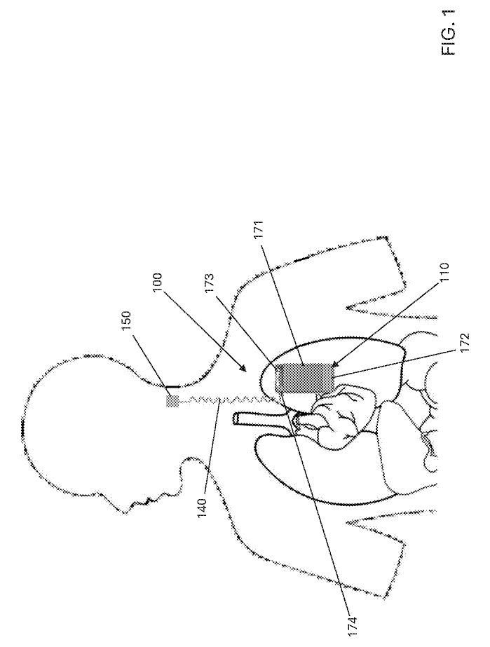

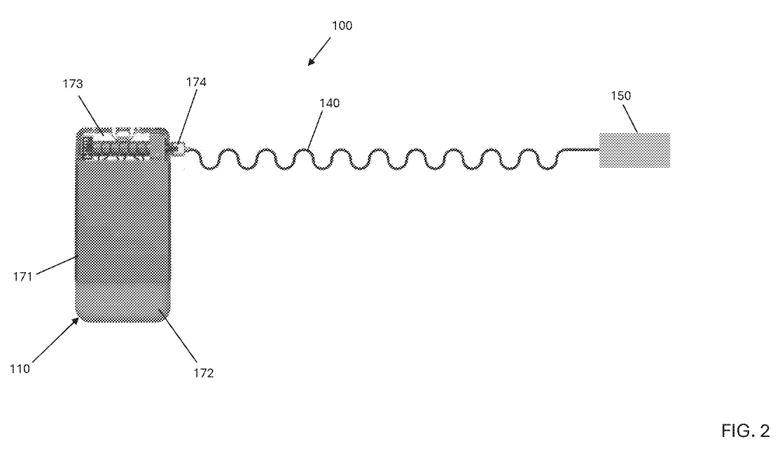

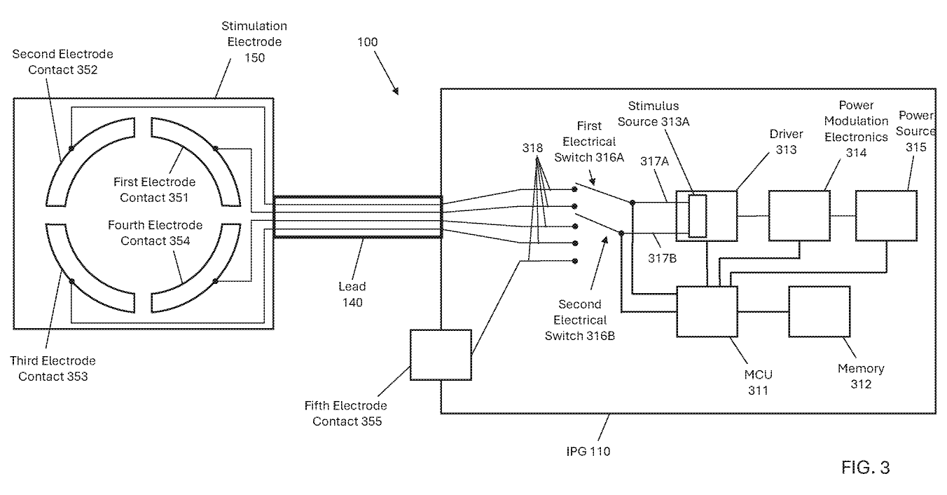

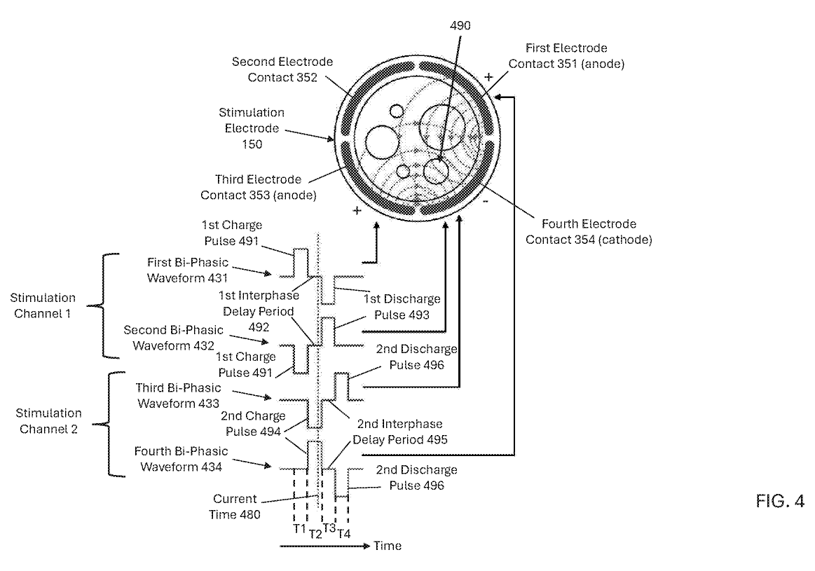

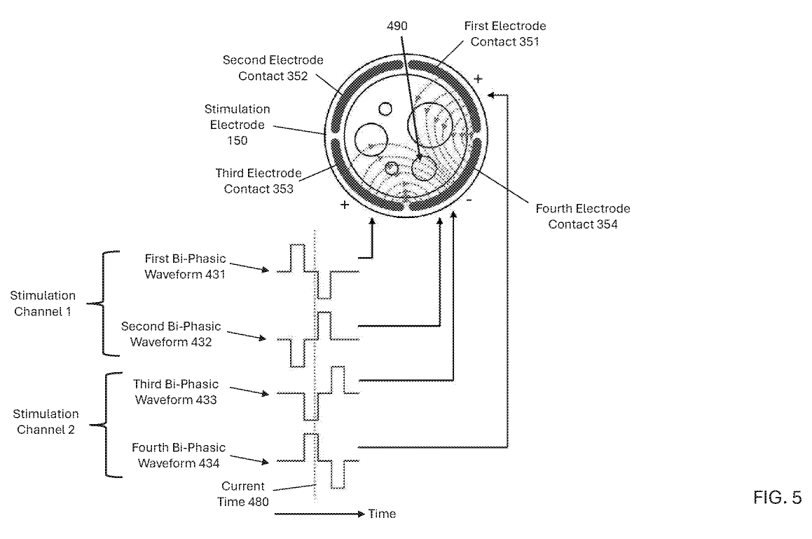

FIG. 1 illustrates an implantable medical system (IMS) 100, according to some examples, when implanted in a patient. FIG. 2 illustrates the IMS 100 of FIG. 1, according to some examples. FIG. 3 illustrates a schematic of the IMS 100 of FIG. 1, according to some examples. FIG. 4 illustrates a cross-sectional schematic of a nerve and a stimulation electrode 150 of the IMS 100 of FIG. 1 according to some examples, stimulus waveforms applied to certain electrode contacts of the stimulation electrode 150 according to some examples, and two electric fields generated by the certain electrode contacts. FIG. 5 illustrates FIG. 4 with the two electric fields superimposed into the combined electric field.

Referring to FIGS. 1-3, the IMS 100 may be configured to be implanted in a human patient or in a non-human animal. For example, the IMS 100 may include biocompatible material(s), and at least some electronic components of the IMS 100 may be hermetically sealed from the exterior of the IMS 100 to prevent or reduce potentially harmful interactions between biological tissue and electronic components of the IMS 100. The IMS 100 may be configured to provide stimulation (e.g., electrical stimulation) to treat one or more medical conditions, such as epilepsy, depression, and/or other medical conditions. For example, the IMS 100 may be configured to stimulate the vagus nerve, the hypoglossal nerve, and/or other biological tissue (e.g., nerve tissue, organ tissue, etc.).

The IMS 100 may include an implantable pulse generator (IPG) 110, a stimulation lead 140 electrically coupled to the IPG 110, and a stimulation electrode 150 on the lead 140. The IPG 110 may be configured to provide electrical stimulus to the stimulation electrode 150 through the lead 140, and the stimulation electrode 150 may be configured to provide the electrical stimulus to tissue around the stimulation electrode 150.

The stimulation electrode 150 is illustrated and primarily described herein as being a nerve cuff electrode that includes four separately chargeable electrode contacts and that is configured to wrap around a nerve such that the electrode contacts at least partially circumferentially surround the nerve. The four electrode contacts include a first electrode contact 351, a second electrode contact 352, a third electrode contact 353, and a fourth electrode contact 354, and the four electrode contacts are illustrated in a wrapped state in FIGS. 3-5. In some other examples, the nerve cuff electrode can have any number of two or more electrode contacts that are configured to be wrapped at least partially circumferentially around a nerve.

As used herein, electrode contacts that are separately chargeable may refer to how each electrode contact can be turned on (e.g., selectively charged as a cathode or as an anode) independently of whether and how the other electrode contacts are turned on. For example, two separately chargeable electrode contacts may be configured such that one of the two electrode contacts can be turned on (e.g., selectively charged as an anode or as a cathode), and the other one of the two electrode contacts can be selectively kept off or turned on (e.g., selectively charged as an anode or as a cathode).

The stimulation electrode 150 is not limited to nerve cuff electrodes and can be any other type of stimulation electrode that includes a plurality (e.g., two or more) electrode contacts and that is configured to stimulate specific tissue. For example, the stimulation electrode 150 may include a paddle electrode having a two-dimensional array of electrodes contacts, a linear or percutaneous electrode having a linear array of electrode contacts placed at the distal end of a stimulation lead, etc.

The configuration of the stimulation electrode 150 for stimulating specific tissue may include the number, shape, and arrangement of electrode contacts. For example, a nerve cuff electrode configured to stimulate the vagus nerve may include a plurality of electrode contacts on a flexible band that is configured to be wrapped around a large nerve such as the vagus nerve. Using a nerve cuff electrode as described herein can be advantageous in that it allows current steering through the nerve with a high degree of control so that specific nerve fascicles can be targeted for activation in a manner that other types of stimulation electrodes cannot effectuate.

The IPG 110 may contain various electronic components and, in most examples, may hermetically seal at least some of these electronic components. The IPG 110 may include a housing or CAN 171, a ceramic base 172 at one end of the CAN 171, and a header 173 at another end (e.g., an opposite end) of the CAN 171.

The CAN 171 may include a metal (e.g., titanium) shell that is configured to contain (e.g., house), and hermetically seal in some examples, at least some of the various electronic components.

The header 173 may include a receptacle 174 configured to receive a proximal end of the lead 140. The header bmay include a plurality of conductive pins configured to electrically connect with corresponding electrical contacts in the lead 140 when the proximal end of the lead 140 is inserted into the lead receptacle 174. The electrical contacts in the lead 140 may be respectively electrically coupled to the electrode contacts in the stimulation electrode 150, and the plurality of conductive pins in the header 173 may be electrically coupled or couplable to electronic components of the IPG 110, for example, to a driver 313 of the IPG 110. The header 173 typically comprises a biocompatible, implantable polymer such as epoxy or polysulfone, which contains the lead receptacle 174.

The ceramic base 172 may be included so that the one end of the CAN 171 is not enclosed by a conductive material. This ceramic base can enable certain magnetic fields (e.g., a charging field for recharging a battery and/or a communication field transmitted to or from the IPG 110) to more easily propagate through the ceramic and into the IPG 110 or out from the IPG 110. However, in some other examples, the ceramic base 172 is omitted, and the CAN 171 is a metal shell or housing, for example, made from a titanium alloy.

The IPG 110 may include a power source 315 (e.g., a rechargeable or non-rechargeable battery), the driver 313, power modulation electronics 314 coupled between the power source 315 and the driver 313, a first electrical switch 316A electrically coupled to the driver 313 via a first electrical path 317A, a second electrical switch 316B electrically coupled to the driver 313 via a second electrical path 317B separate from the first electrical path 317A, a fifth electrode contact 355, a memory 312, and a microcontroller unit (MCU) 311 (e.g., a microcontroller circuit). The IPG 110 may also include other electronic components, such as a wireless transceiver unit and/or one or more sensors.

The wireless transceiver unit may include a receiver and a transmitter, which may be separate or integrally formed into a single unit, and the wireless transceiver may be configured to allow the IPG 110 to wirelessly communicate with devices (e.g., a patient controller, a recharger device, and/or a clinician programmer) outside of the patient. The one or more sensors may be configured to sense one or more physiological conditions of the patient that the IMS 100 is implanted in, which may be used to adjust the stimulation treatment provided to the patient.

The MCU 311 may be operatively connected to at least one of the driver 313, the power modulation electronics 314, the power source 315, the fifth electrode contact 355, the memory 312, the first electrical switch 316A, or the second electrical switch 316B. The MCU 311 may be configured to control at least some operations of the of the IMS 100, including any combination of one or more of the operations of the IMS 100 described herein. The memory 312 may store instructions (e.g., computer readable instructions) that, when accessed and executed by the MCU 311, cause the MCU 311 to perform (e.g., via one or more of the components of the IPG 110) any combination of one or more of the operations of the IPG 110 described herein.

The fifth electrode contact 355 may be separated from the stimulation electrode 150 and may be referred to herein as a distant electrode contact. The fifth electrode contact 355 is often the metal housing 171 of the IPG 110. When the metal housing is used as the distant electrode contact, the metal housing may be referred to as an “indifferent electrode”, “return electrode” or “return anode”. When the metal housing is used as the return anode and at least one of the electrode contacts on the stimulation electrode is used as the cathode electrode contact, this mode of stimulation may be referred to as “unipolar stimulation” or “monopolar stimulation.” Alternatively, stimulation is referred to as “bipolar stimulation” if at least one of the electrode contacts of the stimulation electrode (e.g. a nerve cuff electrode) functions as the cathode electrode contact and at least one electrode contact of the stimulation electrode functions as the anode electrode contact as part of a closed electrical circuit. Because the fifth electrode contact 355 is separated from the stimulation electrode 150, the electric field and electric current propagating through the tissue near the stimulation cathode electrode contact operating in a unipolar mode may be more diffuse compared to the electric field and electric current propagating through tissue near at least two electrode contacts in the stimulation electrode 150 that are operating in the bipolar stimulation mode. The electrical current flow pattern with bipolar stimulation is less diffuse, with the highest electrical current flow being located directly between the cathode and anode electrode contacts, as it is the shortest path and the path of least electrical resistance.

The fifth electrode contact 355 may be a discrete electrode contact that is placed on the IPG 110, instead of the entire metal housing functioning as the fifth electrode contact. For example, the fifth electrode contact 355 may be a dedicated electrode contact attached to, or integrally formed with, the IPG 110. In some examples, part of the IPG 110 is configured to be used as the fifth electrode contact 355. For example, at least part of the CAN 171 and/or at least part of another conductive outer portion of the IPG 110 may be configured to also function as the fifth electrode contact 355. Although the fifth electrode contact 355 will be primarily described in the singular form and as being on the IPG 110, the present disclosure encompasses other examples. For example, the fifth electrode contact 355 may be on a second lead separate from the lead 140, or the fifth electrode contact 355 may be on the lead 140 but separated along the lead 140 from the stimulation electrode 150. The IMS 100 may include a plurality of distant electrode contacts separated from the stimulation electrode 150, and each of the plurality of distant electrode contacts may independently be configured and positioned in any manner that the fifth electrode contact 355 may be configured and positioned in.

The driver 313 may be configured to generate electrical stimulus (e.g., electric current) and may be configured to selectively and differentially apply the electrical stimulus to the electrode contacts of the IMS 100 (e.g., to any combination of the first to fifth electrode contacts 351-355), as described below in more detail. Applying electrical stimulus to an electrode contact may include providing an electric current to the electrode contact to cause a charge to be temporarily stored on the electrode contact. The driver 313 may be configured to generate (e.g., under the control of the MCU 311) the electrical stimulus based on a current (e.g., constant current) or voltage (e.g., constant voltage) provided by the power modulation electronics 314. The power modulation electronics 314 may be configured to modulate (e.g., under the control of the MCU 311) a voltage or current provided by the power source 315 and to provide the modulated voltage or current to the driver 313.

The electrical stimulus generated by the driver 313 may include one or more electrical stimulus pulses to charge or discharge an electrode contact. Charging an electrode contact may include applying electrical stimulus to the electrode contact to cause the electrode contact to transition from an uncharged (off or electrically disconnected) state to functioning as an anode (e.g., an electrode contact storing a positive charge) or as a cathode (e.g., an electrode contact storing a negative charge). For example, charging an electrode contact may include applying electrical stimulus to store a cathodic (negative) charge or an anodic (positive) charge on the electrode contact. An uncharged electrode contact may refer to an electrode contact that has substantially no electrical stimulus applied to it. For example, the uncharged electrode contact may be substantially charge neutral (e.g., having no charge or having a residual charge of a magnitude less than 10%, 5%, 3%, or 1% of the charge previously held by the charged electrode contact). Discharging an electrode contact may include applying electrical stimulus to substantially remove or discharge the cathodic charge or the anodic charge from the electrode contact. For example, discharging an electrode contact may include applying to the electrode contact electrical stimulus having a polarity opposite to electrical stimulus already applied to the electrode contact and having an amplitude substantially equal to (e.g., within 10%, 5%, 3%, or 1% of being exactly equal) the amplitude of the electrical stimulus already applied to the electrode contact.

The driver 313 may be electrically couplable to each of the first to fifth electrode contacts 351-355 through conductive paths 318 (e.g., conductive wires or conductive traces), for example, through separate and respective conductive paths. The conductive paths 318 may be insulated from each other within the IMS 100. The driver 313 may be selectively couplable to each of the conductive paths 318 via first and second electrical switches 316A and 316B, which may be separately electrically coupled to the driver 313 and separately controllable via the MCU 311. For example, the first and second electrical switches 316A and 316B may be respectively electrically coupled to the driver 313 through first and second conductive paths 317A and 317B. In some examples, the first and second conductive paths 317A and 317B are insulated from each other within the IMS 100 and are fixedly coupled between the driver 313 and the first and second electrical switches 316A and 316B.

In some embodiments, the first and second electrical switches 316A and 316B may be respectively electrically coupled (e.g., via the first and second conductive paths 317A and 317B) to the driver 313. In some examples, as shown in FIG. 3, driver 313 may comprise only a single stimulus source 313A. For example, the stimulus source 313A may be the only stimulus source that the driver 313 (or the IPG 110 or IMS 100) has for outputting electrical stimulus. This single stimulus source 313A may be a single direction current source or a single direction voltage source. Using one or more switches of the driver 313, this single stimulus source 313A (e.g., single current source) can be used to drive current in either direction in a closed circuit. For simplicity, we will view the driver 313 as being configured to functionally drive current in either direction in a closed circuit. For example, the one-directional, single stimulus source 313A may be configured to functionally drive current bi-directionally via one or more very fast acting switches and appropriate electrical circuitry of the driver 313. In a bipolar mode of stimulation, the driver 313 can be electrically connected to at least one cathode electrode contact and at least one anode electrode contact that is selected from among the first electrode contact 351, the second electrode 352, the third electrode contact 353, and the fourth electrode contact. In a unipolar mode of stimulation, the driver 313 must be electrically connected to the fifth electrode contact 355 as the return anode electrode and at least one of the first, second, third or fourth electrode contacts 351-354 as the cathode. These electrode contact selections can be made by appropriately switching first electrical switch 316A and second electrical switch 316B to provide a closed electrical circuit connection that includes one anode electrical contact and one cathode electrode contact. A closed circuit, which runs from driver 313 to one electrode contact of the IMS 100, from the one electrode contact to another electrode contact of the IMS 100 and through biological tissue around the IMS 100, and from the other electrode contact to the driver 313 may be formed when the driver 313 drives electrical stimuli, which may be constant current or constant voltage. In some examples, the IMS 100 includes only one driver 313, which may include, in some examples, a single direction current source (e.g., is configured to provide electrical current in one direction at a time), which can be configured to be functionally bi-directional by using one or more appropriate switches (not shown) of the driver 313 to reconfigure the electrical circuit to connect the single direction current source in either direction in a stimulation circuit.

Each of the first and second electrical switches 316A and 316B may be movable between a plurality of positions, whereby the electrical switch is respectively electrically coupled to each of the first to fifth electrode contacts 351-355, for example, through a corresponding one of the conductive paths 318. For example, the first electrical switch 316A may be configured to be moved between a first position, whereby the first electrical switch 316A is electrically coupled to the first electrode contact 351, a second position, whereby the first electrical switch 316A is electrically coupled to the second electrode contact 352, a third position, whereby the first electrical switch 316A is electrically coupled to the third electrode contact 353, a fourth position, whereby the first electrical switch 316A is electrically coupled to the fourth electrode contact 351, and a fifth position, whereby the first electrical switch 316A is electrically coupled to the fifth electrode contact 355. The second electrical switch 316B may be configured in a similar manner as the first electrical switch 316A is configured. The first and second electrical switches 316A and 316B may be movable independently of each other. For example, the first electrical switch 316A may be moved to a different position while the second electrical switch 316B is maintained in its current position or else moved to a new position independently of which position the first electrical switch 316A was moved to. To summarize, the first electrical switch 316A can electrically connect electrical path 317A to first electrode contact 351, second electrode contact 352, third electrode contact 353, fourth electrode contact 354, or to the fifth electrode contact 355. The second electrical switch 316B can electrically connect conductive path 317B to any of the electrode contacts, 351, 352, 353, 354 and 355. If the fifth electrode contact 355, which may be part of the IPG 110 housing, is connected to the stimulation circuit, that may constitute a monopolar/unipolar mode of stimulation. If at least two electrode contacts are selected in the stimulation circuit from contacts 351, 352, 353, and 354, but not 355, that constitutes a bipolar mode of stimulation.

The IPG 110 may be configured to apply electrical stimulus to a pair of electrode contacts by respectively electrically coupling the first and second electrical switches 316A and 316B to the pair of electrode contacts (e.g., to conductive paths 318 respectively electrically coupled to the pair of electrode contacts). The driver 313 can then provide anodic electrical stimulus to one of the pair of electrode contacts and cathodic electrical stimulus to the other one of the pair of electrode contacts. The anodic electrical stimulus and cathodic electrical stimulus are provided concurrently (e.g., simultaneously) such that an electric current flows through the nerve between the pair of electrode contacts as the anodic and cathodic electrical stimuli are respectively provided to the pair of electrode contacts. In some examples, the anodic electrical stimulus may include an anodic electric current that applies an anodic charge to the one electrode contact, and the cathodic electrical stimulus may include a cathodic electric current that applies a cathodic charge to the other one electrode contact.

Applying the electrical stimulus to the pair of electrode contacts may be used, for example, to charge one or both of the electrode contacts (e.g., to charge the one electrode contact as an anode and to charge the other one electrode contact as a cathode). In some examples, the electrical stimulus may be used to discharge one or both of the electrode contacts (e.g., to transition the electrode contact(s) to a substantially charge neutral state). For example, if anodic electrical stimulus is applied to an electrode contact that is electrically charged as a cathode, then the anodic electrical stimulus may substantially discharge the cathodic charge on the cathode to leave the electrode contact substantially charge neutral. In some examples, the electrical stimulus may be used to increase the charge already applied to one or both of the electrode contacts. For example, if cathodic electrical stimulus is applied to an electrode contact that was already charged as a cathode, the cathodic electrical stimulus may increase the cathodic charge stored on the electrode contact.

The IPG 110 (e.g., the MCU 311) may be configured to sequentially apply electrical stimulus to anode-cathode pairs of electrode contacts and then sequentially remove the applied electrical stimulus from the anode-cathode pairs of electrode contacts, for example, in the same sequence by which the electrical stimulus was applied. This may be performed, for example, via a time-multiplexing operation by which one or both of the first and second electrical switches 316A and 316B are moved through a series of different positions while the driver 313 sequentially applies the electrical stimulus and then sequentially removes the applied electrical stimulus. A single driver 313 may therefore be used to selectively and differentially apply electrical stimulus to some or all of the electrode contacts of the IMS 100 to create an electric field and an electric current in the biological tissue around the IMS 100 with a high degree of control over the shape and direction of the electric field and the electric current. This can enable the IMS 100 to target specific tissue (e.g., via current steering) for stimulation (e.g., for activation, in the case of a nerve fascicle being targeted) by controllably concentrating the generated electric field and electric current in the targeted tissue. Because only one driver/current source (or a limited number of drivers) is used instead of a large number of drivers/current sources, the number of components of the IMS 100 (e.g., the number of drivers and their associated electronics) can be reduced, the cost and complexity of operation of the IMS 100 can be reduced, and the size of the IMS 100 can be reduced.

In more detail according to some examples, the first and second electrical switches 316A and 316B may be positioned to electrically couple the driver 313 to a first anode-cathode pair of electrode contacts, and the driver 313 may apply electrical stimulus to this first anode-cathode pair of electrode contacts. Then one or both of the first and second electrical switches 316A and 316B may be repositioned to electrically couple the driver 313 to a second anode-cathode pair of electrode contacts, and the driver 313 may apply electrical stimulus to the second anode-cathode pair of electrode contacts. Electrical stimulus may be applied to each of the first anode-cathode pair of contacts and the second anode-cathode pair of contacts, in a sequential, time-multiplexed manner. A directionally unique electric field and electric current may propagate through the nerve (or other tissue) after electrical stimulus has been applied to both first and second anode-cathode pairs, so that specific nerve fascicle(s) (or other specific tissue) can be targeted for stimulation by the stimulation electrode 150. This can give the IMS 100 a high degree of control over the shape of the electric field and electric current propagating through the nerve or other biological tissue so that the IMS 100 has increased control over which nerve fascicles (or other specific tissue) is targeted.

Example waveforms of biphasic electrical stimulus, using bipolar mode of stimulation, are illustrated in FIGS. 4 and 5 together with the resulting electric field generated in the nerve that the nerve cuff electrode 150 circumferentially surrounds. Both FIGS. 4 and 5 show a cross-sectional representation of a nerve cuff stimulation electrode 150 having four electrode contacts, including a first electrode contact 351, a second electrode contact 352, a third electrode contact 353, and a fourth electrode contact 354, within the cuff. The cuff encircles a nerve bundle, shown cross-sectionally, with individual nerve fascicles, including a target nerve fascicle 490. The horizontal axis of the stimulus waveforms represents time, with the current time being represented by reference number 480 in FIGS. 4-5. The vertical axis of the waveforms represents the amplitude of the electrical stimulus being applied (e.g., amplitude of the electric current). As shown in the non-limiting examples of FIGS. 4-5, two biphasic stimulus waveforms are provided. A first biphasic stimulus waveform includes first and second biphasic waveforms 431 and 432, representing the stimulus applied to a first anode-cathode pair (electrode anode contact 353 and electrode cathode contact 354) operating as a complete electrical circuit and labeled as Stimulation Channel 1 operating in a bipolar stimulation mode. For purposes of this disclosure, an electrode contact (e.g., 354) will be labeled a cathode, when a biphasic stimulus waveform (e.g., 432) has a negative, cathodic, first phase or first charge pulse, even if the biphasic stimulus waveform (e.g., 432) has a positive discharge 493. Conversely, if the first charge pulse (e.g., 491) of a biphasic stimulus waveform (e.g., 431), which is delivered through an electrode contact (e.g., 353) has a positive phase, that electrode contact (e.g., 353) will be labeled as an anode. Hence, the electrode contact will be categorized as functioning as a cathode or anode in accordance with the polarity of the delivered first charge pulse of a biphasic stimulus pulse or waveform. Similarly, a second biphasic stimulus waveform includes third and fourth biphasic waveforms 433 and 434, representing the stimulus applied to a second anode-cathode pair (electrode anode contact 351 and electrode cathode contact 354) operating as a complete electrical circuit in bipolar stimulation mode and labeled as Stimulation Channel 2. Note that in this embodiment, cathode contact 354 remains the same for both Stimulation Channel 1 and Stimulation Channel 2.

In stimulus waveforms, 432, 433 the negative first pulse and positive second pulse have opposite polarities and can be separated by an interphase delay period 492, 495. Also, in stimulus waveforms, 431, 434, the positive first pulse and negative second pulse have opposite polarities and can be separated by an interphase delay period 492, 495. For example, the first biphasic stimulus waveform 431 includes a first charge pulse 491 (a positive, anodic pulse) and a first discharge pulse 493 (a negative, cathodic pulse), which have opposite polarities and are separated in time by a first interphase delay period 492. The second biphasic stimulus waveform 432 includes a first charge pulse 491 (a negative, cathodic pulse) and a first discharge pulse 493 (a positive, anodic pulse), which have opposite polarities and are separated in time by the first interphase delay period 492. In the first stimulation channel 1, the first charge pulses 491, the first interphase delay period 492, and the first discharge pulses 493 respectively sequentially occur during a first time duration T1, a second time duration T2, and a third time duration T3. The third biphasic stimulus waveform 433 includes a second charge pulse 494 (a negative, cathodic pulse) and a first discharge pulse 496 (a positive, anodic pulse), which have opposite polarities and are separated in time by a second interphase delay period 495. The fourth biphasic stimulus waveform 434 includes a second charge pulse 494 (a positive, anodic pulse) and a first discharge pulse 496 (a negative, cathodic pulse), which have opposite polarities and are separated in time by the first interphase delay period 492. In the second stimulation channel 2, the second charge pulses 494, the second interphase delay period 495, and the second discharge pulses 496 respectively sequentially occur during the second time duration T2, the third time duration T3, and a fourth time duration T4.

The discharge pulse 493, 496 of a biphasic stimulus waveform may have parameters (e.g., amplitude, time duration, and/or shape) set to substantially remove (e.g., discharge or neutralize) the electrical stimulus applied to the electrode contact via the charge pulse 491, 495 of the same biphasic waveform. In the depicted example of FIGS. 4-5, the discharge pulses have an opposite polarity as the corresponding charge pulse and have the same amplitude, shape, and time duration. However, in other embodiments of stimulus waveforms, the discharge pulse may have a different shape than the charge pulse, although preferably with areas within the discharge pulse and charge pulse about equal so that excess charge does not accumulate at the cathode contact and cause accelerated contact corrosion. This is referred to as “charge balancing.”

The stimulus waveforms applied at the cathode-anode pair may be identical to each other except for their polarities. For example, first stimulation channel 1 includes cathode contact 354 and anode contact 353. The corresponding stimulus at the cathode contact 354 and anode contact 353 is provided by stimulus waveforms 432 and 431, respectively. A second stimulation channel includes cathode contact 354 and anode contact 351. Opposite polarity pulses in the same stimulation channel may be applied concurrently (e.g., simultaneously) to the anode-cathode pair by the driver 313 through the first and second electrical switches 316A and 316B.

As shown in FIG. 4, the sequence of stimulation, in bipolar mode of stimulation, occurs in the following steps. In time duration T1, the first anode-cathode pair of electrode contacts 353 and 354 are electrically connected with switches (e.g. 316A, 316B). First charge pulse 491 is applied at cathode 354 and the opposite charge pulse is applied to anode 353. Then, during T2, the switches or in this case one switch connects anode 351 and thereby electrically connects second anode-cathode pair of electrode contacts 351, 354. A second charge pulse 494 is applied to cathode 354 and an opposite polarity charge pulse is applied to anode 351. During T3, anode 351 is disconnected and anode 353 is connected and thereby first anode-cathode pair, 353, 354 are electrically connected to the driver 313 via the switches. A first discharge pulse 493 is applied to the cathode 354 and an opposite polarity discharge pulse is applied at anode 353. During T4, anode 353 is disconnected and anode 351 is connected so that the second anode-cathode pair, 351, 354 are electrically connected to the driver 313 via the switches. A second discharge pulse is applied at cathode 354 and an opposite polarity discharge pulse is applied at anode 351. The amplitudes of the first charge pulse 491 and second charge pulse 494 (either delivered as a constant current amplitude or a constant voltage amplitude) may be different or the same. When the amplitude of the first charge pulse 491 and the amplitude of the second charge pulse 494 are different, the current flow will steer towards the anode contact with the higher amplitude stimulus charge pulse.

In some examples, the charge pulse of a biphasic waveform may be applied during the interphase delay period of at least one of (e.g., each of) the biphasic waveform (s) whose charge pulses have previously been applied. The discharge pulse of a biphasic waveform may be applied during the interphase delay period of at least one of (e.g., each of) the biphasic waveform(s) whose discharge pulses have not yet been applied. For example, the charge pulses of the third and fourth biphasic waveforms 433 and 434 are applied during the first interphase delay period 492 of the first and second biphasic waveforms 431 and 432, and the discharge pulses of the first and second biphasic waveforms 431 and 432 are applied during the second interphase delay 495 period of the third and fourth biphasic waveforms 433 and 434. This process may be implemented, for example, by a time-multiplexing operation whereby, after the driver 313 applies the charge pulses for one pair of biphasic waveforms, one or both of the first and second electrical switches 316A and 316B is moved to a new position and the driver 313 applies the charge pulses for a next pair of biphasic waveforms in the sequence. A similar time-multiplexing operation may be implemented to sequentially apply the discharge pulses.

Providing an interphase delay period between the charge and discharge pulses of a pair of biphasic electrical stimulus pulses can allow the electric field and electric current between the charged electrode contacts to build up and stabilize after the charge pulses are applied and before the discharge pulses are applied. This can improve the stimulation of the target tissue compared to if the discharge pulse is immediately applied following the charge pulse before the electric field and electric current have sufficient time to build up and trigger an action potential in the targeted tissue.

By using the same driver to apply one or more other charge pulses to one or more other electrode contacts during this interphase delay period, a single driver (e.g., a single current source) can sequentially charge a plurality of different electrode contacts before discharging the electrode contacts and without requiring a large number of dedicated drivers (e.g., dedicated current sources). When these electrode contacts are all charged, they will generate a plurality of electric fields, which will superimpose to form a combined electric field having a unique shape and direction based on which electrode contacts are charged and the amplitudes and polarities of the applied electrical stimulus. This combined electric field can then apply stimulation (e.g., generate an action potential) in the targeted tissue before the charged electrode contacts are discharged. Thus, a single driver can implement current steering through biological tissue without requiring multiple drivers (e.g., multiple current sources). This can reduce the cost, number of components (e.g., number of drivers and associated electronics), complexity of operation, and size of the IMS 100 compared to if a large number of drivers were included in the IMS 100.

FIG. 4 illustrates, within the nerve cuff stimulation electrode 150, two electric fields respectively generated by the third and fourth electrode contacts 353 and 354 and by the first and fourth electrode contacts 351 and 354 at a time 480 at which the charge pulses of the first to fourth biphasic waveforms 431-434 have been applied and before the discharge pulses of the first to fourth biphasic waveforms 431-434 have been applied. The superposition of these two electric fields is shown in FIG. 5. Because the fourth electrode contact 354 (functioning as a cathode) has been charged twice (once by the first charge pulse 491 and a second time by the second charge pulse 494), the electric field lines are more densely grouped in the medium-sized target nerve fascicle 490 that is near the cathode (the fourth electrode contact 354 in this example) compared to if only two of the four electrode contacts were charged. Accordingly, electric current flowing through the nerve is steered toward the target nerve fascicle 490 to activate this particular nerve fascicle. It can be desirable to target nerve fascicles for activation based on their size. For example, mid-size and larger nerve fascicles typically can be stimulated/activated at a lower stimulus amplitude than a smaller diameter nerve fascicle.

The present disclosure is not limited to examples where only one driver is included in the IMS. For example, an IMS may include two drivers or a limited number of drivers, each being configured to sequentially charge a plurality of electrode contacts in any manner described herein (e.g., via a time-multiplexing operation), and the IMS may still have reduced cost, number of components, complexity of operation, and size compared to if a large number of drivers (e.g., dedicated drivers that are not configured to perform a time-multiplexing operation) were included in the IMS.

This process of charging and discharging a plurality of electrode contacts via a plurality of sequentially applied pairs of biphasic waveforms may be repeatedly performed, for example, at a set frequency and/or with a set duty cycle, in order to repeatedly activate the same target tissue (e.g., target nerve). For example, in the example of FIGS. 4-5, the first pair of biphasic waveforms (including the first and second biphasic waveforms 431 and 432) and the second pair of biphasic waveforms (including the third and fourth biphasic waveforms 433 and 434) may be alternatingly applied to repeatedly activate the target nerve fascicle 490.

FIGS. 4-5 illustrated a non-limiting example of bipolar stimulation where two pairs of biphasic waveforms are applied by the driver 313 to electrode contacts in the stimulation electrode 150. However, other examples are within the scope of the present disclosure.

For example, the anode-cathode pair may include a fifth electrode contact 355 functioning as an anode and that is separated from the stimulation electrode e.g. nerve cuff. When the fifth electrode contact 355 is part of the IPG housing, this is a unipolar or monopolar mode stimulation. The fifth electrode functions as a return anode and typically has a much larger surface area compared to the electrode contacts in the stimulation electrode 150. The large surface area of the fifth electrode/return anode presents a diffuse current/charge density and thereby prevents inadvertent stimulation of tissue surrounding the fifth electrode and IPG.

In some examples, the IMS 100 may be configured to only charge multiple electrode contacts in the stimulation electrode 150 as cathodes and to use the fifth electrode contact 355 as the anode. For example, two or more of the first to fourth electrode contacts 351-354 may be charged as cathodes, and the fifth electrode contact 355 may be charged as the return anode.

In some other examples, such as in the example of FIGS. 4-5, electrode contacts in the stimulation electrode 150 are charged both as anode(s) and as cathode(s), and one of the cathode(s) may have an amplitude of cathodic electrical stimulus applied to it that is greater than the amplitude(s) of anodic electrical stimulus applied to the anode(s). The cathode having the high amplitude of charge may be selected to be nearer to the target tissue than the other charged electrode contacts so that the generated electric field may be strongest near the target tissue and so that the target tissue will be activated by said cathode.

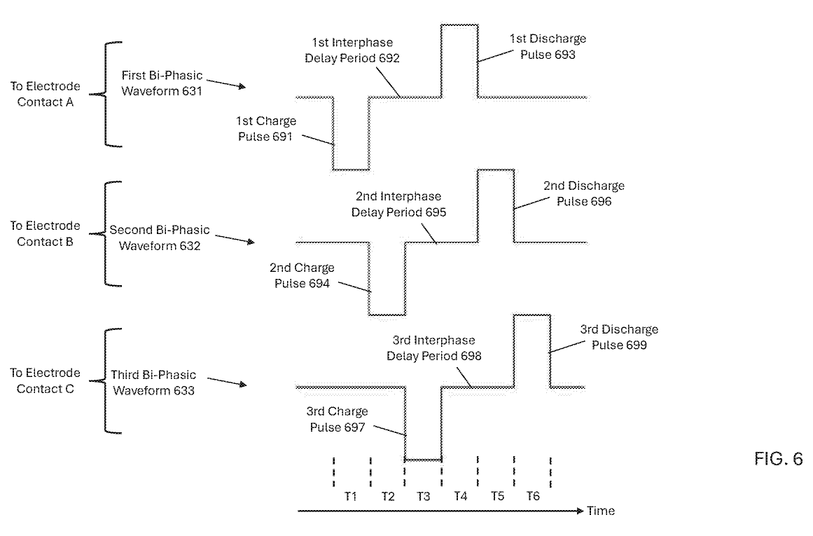

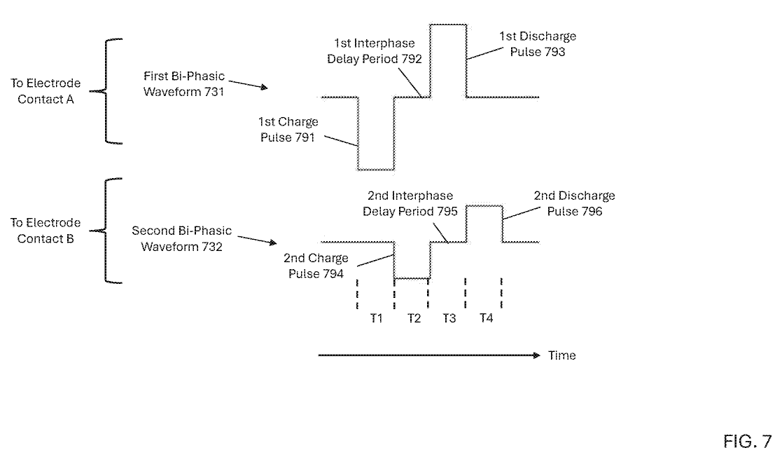

FIGS. 6-10 each illustrate waveforms of a biphasic stimulus waveform according to some other examples in a unipolar stimulation mode. For ease of illustration, in each of FIGS. 6-10, each of the illustrated biphasic stimulation waveforms will show what is applied by the IPG output circuitry to each of 3 cathode electrode contacts A, B, C (FIG. 6) or 2 cathode electrode contacts A and B (FIGS. 7-10). The corresponding stimulation return anode waveform is not shown. However, it will be understood that the anode waveform will mirror and have opposite polarity to the cathode waveform.

Referring to FIG. 6, in some examples, in a unipolar stimulation mode, three or more pairs of biphasic stimulus waveforms may be sequentially applied to three separate cathode electrode contacts A, B, and C, in a similar time-multiplexing manner as described herein. In FIG. 6, three biphasic stimulus waveforms are illustrated, including a first biphasic waveform 631, a second biphasic waveform 632, and a third biphasic waveform 633.

The first biphasic stimulus waveform 631 may include a first cathodic charge pulse 691, a first interphase delay period 692, and a first discharge pulse 693. Similarly, the second biphasic stimulus waveform 632 may include a second cathodic charge pulse 694, a second interphase delay period 695, and a second discharge pulse 696. The third biphasic waveform 633 may include a third charge pulse 697, a third interphase delay period 698, and a third discharge pulse 699. The second biphasic waveform 632 is time-shifted to a later time relative to the first biphasic waveform 631, and the third biphasic waveform 633 is time-shifted to a later time relative to the second biphasic waveform 632.

The first to third biphasic stimulus waveforms 631-633 shown in the example of FIG. 6 may be implemented, in unipolar stimulation configuration in the following sequence. During the time duration T1, the cathode contact A is electrically connected (on), and a first cathodic charge pulse 691 is applied at the cathode electrode contact A. Then in time duration T2, the cathode contact A is electrically disconnected (off) and cathode contact B is electrically connected (on), and a second cathodic charge pulse 694 is applied to cathode contact B. Then in time duration T3, cathode contact B is disconnected (off) and cathode contact C is connected (on), and a third cathodic charge pulse 697 is applied to cathode contact C. In time duration T4, electrode contact C is disconnected (off) and electrode contact A is connected (on), and a first discharge pulse 693 is applied to electrode contact A. Then in time duration T5, electrode contact A is disconnected (off) and electrode contact B is connected (on), and a second discharge pulse 696 is applied to electrode contact B. Then, in in time duration T6, electrode contact B is disconnected (off) and electrode contact C is connected (on), and a third discharge pulse 699 is applied to electrode contact C. This entire sequence of stimulation T1-T6 may be repeated M number of times, which provides a finite time duration for stimulation. This sequential pattern of stimulation can provide current steering using three separate cathode contacts within an electrode, such as a nerve cuff electrode, using time multiplexing, and using a single switch to electrically connect (and disconnect) one of cathode contacts A, B or C. As seen in FIG. 6, in one embodiment, the first, second, and third cathodic, charge pulses 691, 694, and 697 are shown as the same amplitudes. However, each of the first, second and third charge pulses may be different amplitudes to effect current steering. It is preferred that the cathode charge pulse and discharge pulse on each cathode contact be charged balanced. In other words the amplitude of charge pulses 691, 694, and 697 is equal to discharge pulses 693, 696, and 699, respectively, to achieve charge balancing at each respective cathode contact, A, B and C. It will be understood that this time multiplexed, sequential stimulation process can be used, in unipolar stimulation configuration, with just two cathode contacts, three cathode contacts, or four or more cathode contacts, using, for example, the nerve cuff electrode depicted in FIG. 3, a paddle type electrode, or a linear lead with multiple electrode contacts or other electrodes with a plurality of electrode contacts.

It is instructive to describe the operation of the first electrical switch 316A, for example, as shown in FIG. 3, to implement the time multiplexed stimulation sequence of FIG. 6. For unipolar stimulation mode, second electrical switch 316B is always electrically connected to the Fifth Electrode Contact 355 which will be the anode return electrode, typically located at the IPG housing. The first electrical switch 316A, however, will cycle through connection to each of the chosen cathode electrode contacts. For example, the three electrode contacts can be chosen to be cathodes: Contact A is first electrode contact 351, Contact B is second electrode contact 352 and Contact C is third electrode contact 353. Referencing FIG. 6, the switch 316A will be positioned so that it will: (1) in T1, connect to Electrode Contact A to apply a first charge pulse 691 (2) in T2, connect to Electrode Contact B to apply a second charge pulse 694, (3) in T3, connect to electrode contact C to apply a third charge pulse 697, (4) in T4, connect to electrode contact A to apply a first discharge pulse 693, (5) in T5, connect to electrode contact B to apply a second discharge pulse 696, and (6) in T6, connect to electrode contact C to apply a third discharge pulse 699. Note that this stimulation process can be used with two, three, four or more chosen cathode contacts, in a time multiplexed manner to provide current steering in a nerve cuff, paddle, or linear lead.

Applying two or more stimulus waveforms in selected cathodic electrode contacts, in a unipolar stimulation configuration, can increase current steering capabilities of the IMS 100 by increasing the number of different combinations of electrical stimulus that can be applied to the electrode contacts and, thus, increasing the number of differently shaped electric fields that the IMS 100 is configured to generate. This can also enable the IMS 100 to generate a higher strength electrical field through the center region of a nerve bundle to better enable the IMS 100 to target larger nerve fascicles compared to if only one stimulus waveform is applied.

Although FIG. 6 has been described in terms of providing unipolar mode of stimulation according to some examples, where the three anodic stimulation waveforms (not shown), corresponding to the three depicted cathodic stimulation waveforms through cathode electrode contacts A, B and C , are all applied to a distant return anode, such as the IPG housing, the present disclosure also encompasses examples where the stimulation is instead bipolar (not shown). For example, the three anodic stimulation waveforms (not shown) may instead be provided to one or more anode contacts on the stimulation electrode 150 to provide bipolar stimulation.

Referring to FIG. 7, in some examples, the IPG and IMS 100 may be configured in a unipolar mode of stimulation to differentially apply electrical stimulus biphasic waveforms having charge pulses with different first and second stimulus amplitudes using two selected cathode electrode contacts A and B. In this sequence there will be four time durations: a first time duration T1 to connect cathode A and to apply a first charge pulse 791, a second time duration T2- to connect cathode B and to apply a second charge pulse 794, a third time duration T3 to connect cathode A and to apply a first discharge pulse 793, and a fourth time duration T4 to connect cathode B and to apply a second discharge pulse 796. Note that the charge pulse and discharge pulse at each cathode contact are charged balanced. This can increase current steering capabilities of the IMS 100 by increasing the number of different combinations of electrical stimulus that can be applied to electrode contacts of the IMS. In turn, this can increase the number of differently shaped electric fields that the IMS 100 is configured to generate.

FIG. 7 illustrates a first biphasic waveform 731 and a second biphasic waveform 732, which is time-shifted to a later time compared to the first biphasic waveform 731. The first biphasic waveform 731 may include the first charge pulse 791, a first interphase delay period 792, and the first discharge pulse 793. The first charge pulse 791 pulse width may be, in some embodiments, between about 6.2 microseconds and 2 milliseconds. The first discharge pulse 793, may be, in some embodiments, between about 6.2 microseconds and 2 milliseconds. The second charge pulse 794 pulse width may be, in some embodiments, between about 6.2 microseconds and 2 milliseconds. The second discharge pulse 796, may be, in some embodiments, between about 6.2 microseconds and 2 milliseconds. The second biphasic waveform 732 may include the second charge pulse 794, a second interphase delay period 795, and the second discharge pulse 796. The first charge pulse 791 has a first amplitude, and the second charge pulse 794 has a second amplitude less than the first amplitude. In other examples, the second amplitude may be greater than the first amplitude. By applying two charge pulses of different amplitudes respectively to, for example, two different cathode electrode contacts, the combined electric field can be selectively made more concentrated near one of the electrode contacts so that control over which nerve fascicle (or other specific tissue) is targeted is increased compared to if the two charge pulses had equal amplitudes.

Although FIG. 7 has been described in terms of providing unipolar stimulation according to some examples, the present disclosure also encompasses examples where bipolar stimulation is instead provided (not shown).

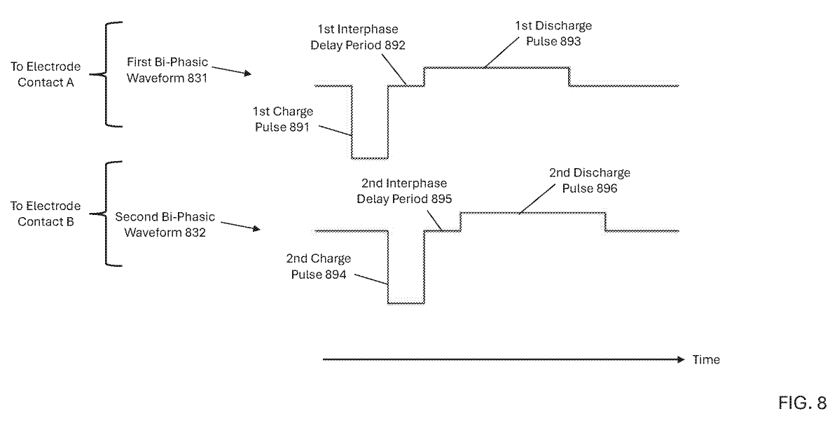

FIG. 8 shows, in a unipolar mode of stimulation, the biphasic stimulus waveforms 831, 832 delivered through cathode electrode contacts A and B, respectively. The time period of the discharge pulse 893, 896 of a biphasic waveform 831, 832 may be different from (e.g., longer than) the time period of the charge pulse 891, 894 of the same biphasic waveform 831, 832, respectively. Extending the discharge period and reducing the amplitude of the discharge pulse 893, 896 to below nerve stimulation threshold can avoid generating an additional and unintended action potential (e.g., of a nerve fascicle that is not being targeted) during the discharge period. In some examples, the pulse width of the first discharge pulse 893 is between about four to sixteen times the pulse width of the first charge pulse 891. In some examples, the pulse width of the second discharge pulse 896 is between about four to sixteen times the pulse width of the second charge pulse 894.

FIG. 8 illustrates, for example, in unipolar mode of stimulation, a first biphasic waveform pulse 831 (at the first cathode electrode contact A) and a second biphasic waveform 832 (at the second cathode electrode contact B). The first biphasic waveform 831 may include a first cathodic (-) charge pulse 891, a first interphase delay period 892, and a first anodic (+) discharge pulse 893. The second biphasic waveform 832, which is slightly delayed in time compared to the first biphasic waveform 831, may include a second cathodic (-) charge pulse 894, a second interphase delay period 895, and a second anodic (+) discharge pulse 896. As shown in FIG. 8, the time durations for the first and second discharge pulses 893 and 896 are longer than the time durations for the first and second charge pulses 891 and 894, respectively. In some examples, the first and second discharge pulses 893 and 896 may overlap in time, which may require the IPG to have at least two drivers so that the first and second discharge pulses 893 and 896 can be simultaneously applied. Alternatively, the discharge pulses 893 and 896 may be applied by a single driver by coupling the driver to two anode electrode contacts and to two cathode electrode contacts in parallel. The amplitudes of the first and second discharge pulses 893 and 896 may be smaller than the amplitudes of the first and second charge pulses 891 and 894 so that, in view of the longer time durations, the first and second discharge pulses 893 and 896 respectively substantially reverse the electrical stimulus applied by the first and second charge pulses 891 and 894. Preferably, the total charge of first cathodic pulse 891 will be equal to the first discharge pulse 893 to balance stimulus (-) charge and discharge (+) charge at the electrode contact. Similarly, the total charge in second charge pulse 894 will be preferably equal to the total charge in second discharge pulse 896. These waveforms are considered to be non-symmetric, charged-balanced, biphasic stimulus waveforms. Charge balancing of a biphasic stimulus waveform is desirable as it prevents accumulation of excess charge and premature corrosion of the electrode contact.

Although FIG. 8 has been described in terms of providing unipolar mode of stimulation according to some examples, the present disclosure also encompasses examples where bipolar stimulation is instead provided (not shown).

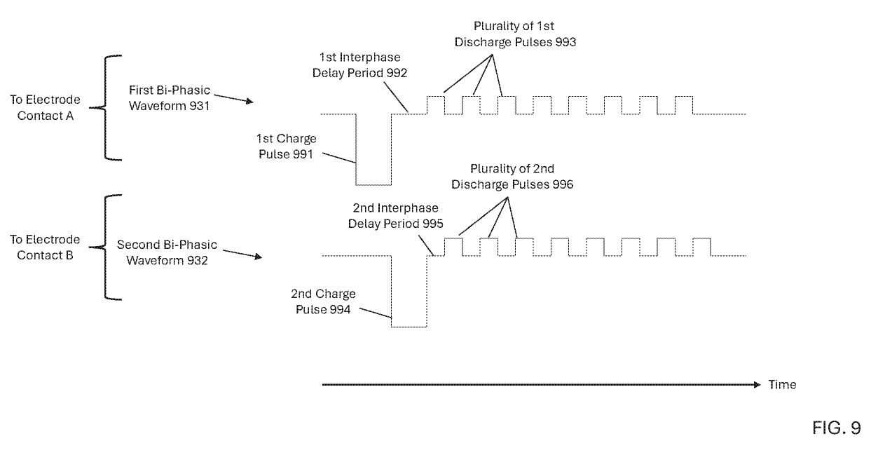

FIG. 9 shows, in unipolar mode of stimulation, in some examples, a biphasic stimulus waveform that may include a plurality of discharge pulses instead of a single discharge pulse. Similar to increasing the time duration of the discharge pulses, as discussed in FIG. 8, discharging the charged electrodes by a plurality of small-amplitude discharge pulses, instead of by a single high-amplitude discharge pulse, can reduce the risk of unintentionally activating a non-targeted nerve fascicle (or other non-targeted tissue) during the discharge phase by reducing the amplitude of the discharge pulses 993, 996. FIG. 9 illustrates a first biphasic waveform 931 and a second biphasic waveform 932 being delivered by cathode electrode contact A and cathode electrode contact B, respectively. The first biphasic waveform 931 may include a first charge pulse 991, a first interphase delay period 992, and a plurality of first discharge pulses 993. To achieve charge balancing, the total charge in charge pulse 991 is preferably equal to the sum of charges represented by all of the first discharge pulses 993. The second biphasic stimulus waveform 932 may include a second charge pulse 994, a second interphase delay period 995, and a plurality of second discharge pulses 996. To achieve charge balancing, the total charge in the second charge pulse 994 is preferably equal to the total charge represented by the sum of the second discharge pulses 996. Each of the pluralities of first and second discharge pulses 993 and 996 may include 2 or more pulses, for example, 3 pulses, 5 or more pulses, or 10 or more pulses.

The parameters (e.g., amplitude and time duration) of a discharge pulse among a plurality of discharge pulses may differ from the parameters of the charge pulse in the same biphasic waveform. For example, the amplitude of the discharge pulse may be smaller than the amplitude of the charge pulse and/or the time period of the discharge pulse may be smaller than the time duration of the charge pulse. The parameters of the plurality of discharge pulses in a biphasic waveform may be set to substantially reverse and charge balance the electrical charge applied to an electrode contact by the charge pulse of the same biphasic waveform stimulus.

The plurality of second discharge pulses 996 may be alternatingly applied with the plurality of first discharge pulses 993. For example, the time period of a discharge pulse from among the plurality of second discharge pulses 996 may be entirely between two adjacent discharge pulses from among the plurality of first discharge pulses 993. This may be implemented, for example, in unipolar stimulation, by toggling an electrical switch (e.g., the first electrical switch 316A or the second electrical switch 316B) between two positions, whereby the electrical switch respectively couples the driver 313 to two different cathode electrode contacts that the pluralities of first and second discharge pulses 993 and 996 are to be applied to. The driver 313 can then alternatingly apply discharge pulses to the two cathode contacts as the electrical switch is toggled between the two cathode contacts. Because the discharge pulses are alternated (instead of overlapping), the two pluralities of discharge pulses can be applied by a single driver, via toggling the electrical switch back and forth.

Although FIG. 9 has been described in terms of providing unipolar stimulation according to some examples, the present disclosure also encompasses examples where bipolar stimulation is instead provided.

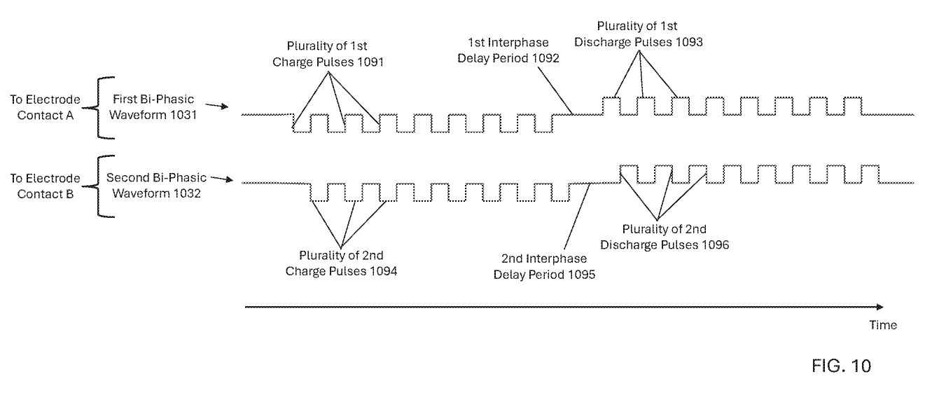

FIG. 10 shows a unipolar mode of stimulation using, in some examples, a biphasic stimulus waveform that may include a plurality of charge pulses instead of a single charge pulse. For example, two biphasic stimulus waveforms 1031, 1032 may each have a plurality of charge pulses 1091, 1094 that are alternatingly applied through cathode electrode contact A and cathode electrode contact B in a similar manner as the first and second plurality of discharge pulses 993 and 996 are alternatingly applied in the example of FIG. 9. The first biphasic waveform 1031 may include a plurality of first charge pulses 1091, a first interphase delay period 1092, and a plurality of first discharge pulses 1093. In some examples, a single pulse of the plurality of first charge pulses 1091 may be between 1% to 10% of the total time duration of the plurality of first charges 1091. In some examples, a single discharge pulse of the plurality of first discharge pulses 1093 may be between 1% to 10% of the total time duration of the plurality of first discharge pulses 1093. In some examples, a single pulse of the plurality of second charge pulses 1094 may be between 1% to 10% of the total time duration of the plurality of second charge pulses 1094. In some examples, a single discharge pulse of the plurality of second discharge pulses 1096 may be between 1% to 10% of the total time duration of the plurality of second discharge pulses 1096. To achieve charge balancing, the sum of charges represented by all of the first charge pulses 1091 may be equal to the sum of charges represented by all of the first discharge pulses 1093. The second biphasic stimulus waveform 1032 may include a plurality of second charge pulses 1094, a second interphase delay period 1095, and a plurality of second discharge pulses 1096. To achieve charge balancing, the sum of charges represented by all of the second charge pulses 1094 may be equal to the sum of charges represented by all of the second discharge pulses 1096.

Each of the pluralities of first and second charge pulses 1091 and 1094 may include 2 or more pulses, for example, 3 pulses, 5 or more pulses, or 10 or more pulses. Each of the pluralities of first and second discharge pulses 1093 and 1096 may include 2 or more pulses, for example, 3 pulses, 5 or more pulses, or 10 or more pulses. The number of first charge pulses 1091 may be the same as, or different from, the number of second charge pulses 1094, and the number of first discharge pulses 1093 may be the same as, or different from, the number of second discharge pulses 1096. The number of first discharge pulses 1093 may be the same as, or different from, the number of first charge pulses 1091, and the number of second discharge pulses 1096 may the same as, or different from, the number of second charge pulses 1094.

The parameters (e.g., amplitude and time duration) of a discharge pulse among a plurality of discharge pulses may be the same as, of different from, the parameters of a charge pulse among a plurality of charge pulses of the same biphasic waveform. For example, the amplitude of one (or each) of the first discharge pulses 1093 may be the same as, or different from (e.g., larger than or smaller than), the amplitude of one (or each) of the first charge pulses 1091 and/or the duration of the one (or each) of the first discharge pulses 1093 may be the same as, or different from (e.g., larger than or smaller than), the duration of the one (or each) of the first charge pulses 1091. Also, the amplitude of one (or each) of the second discharge pulses 1096 may be the same as, or different from (e.g., larger than or smaller than), the amplitude of one (or each) of the second charge pulses 1094 and/or the duration of the one (or each) of the second discharge pulses 1096 may be the same as, or different from (e.g., larger than or smaller than), the duration of the one (or each) of the second charge pulses 1094. The parameters of the charge pulses and discharge pulses of a same biphasic pulse may be set to achieve charge balancing, as discussed above.

The plurality of second charge pulses 1094 may be alternatingly applied with the plurality of first charge pulses 1091. For example, the time period of a charge pulse from among the plurality of second charge pulses 1094 may be entirely between two adjacent charge pulses from among the plurality of first charge pulses 1091. A time period of a charge pulse from among the plurality of first charge pulses 1091 may be entirely between two adjacent charge pulses from among the plurality of second charge pulses 1094. This may be implemented, for example, in unipolar stimulation mode (where the metal conductive portion of the IPG or stimulator CAN or housing functions as a return anode) by toggling an electrical switch (e.g., the first electrical switch 316A or the second electrical switch 316B) between two positions, whereby the electrical switch respectively couples the driver 313 to two different cathode (-) electrode contacts that the pluralities of first and second charge pulses 1091 and 1094 are applied to. The driver 313 can then alternatingly apply charge pulses to the two cathode contacts as the electrical switch is toggled between the two cathode contacts. Because the charge pulses are alternated (instead of overlapping), the two pluralities of charge pulses can be applied by a single driver, via toggling the electrical switch back and forth. Also, the plurality of second discharge pulses 1096 may be alternatingly applied with the plurality of first discharge pulses 1093 in a similar manner as discussed above with respect to the pluralities of first and second discharge pulses 993 and 996 of the example of FIG. 9. In a bipolar stimulation mode, using a plurality of charge pulses can allow for two or more pairs of anode and cathode contacts to be charged by interleaving their respective pluralities of charge pulses. For example, after applying a pair of charge pulses to one pair of anode and cathode contacts, the driver 313 can be switched (via the first electrical switch 316A and/or the second electrical switch 316B) to apply charge pulses to a different pair of anode and cathode contacts. This can allow for a very long cumulative charge pulse width (accounting for all of the charge pulses) to be applied during the charging stage via a plurality of relatively short charge pulses. For example, the total time by which a plurality of charge pulses (e.g., the first charge pulses 1091 or the second charge pulses 1092) may be applied may be within a range of 0.5 ms to 5 ms, 1 ms to 3 ms, or 1 ms to 2 ms. The duration of each charge pulse (e.g., the duration of one of the first charge pulses 1091 or of one of the second charge pulses 1094) may be less than 200 µs, less than 100 µs, or less than 50 µs (e.g., 31 µs).

Using a plurality of charge pulses in each biphasic waveform can be helpful in the case of anode intensification, whereby equal or more charge is applied to two neighboring anode contacts that surround a cathode contact. For example, if one of the electrode contacts (e.g., the fourth electrode contact 354) is charged as a cathode contact with a charge of (q-), each of the two neighboring electrode contacts (e.g., the first electrode contact 351 and the third electrode contact 353) may be charged as anodes respectively with a charge of (q-). This can help to narrow the area of activation on a nerve and make it more selective. This method may also entail delivering the excess charge (q-) to a remote electrode contact, such as the fifth electrode contact 355, for charge balancing. By using the time-multiplexing scheme described herein, whereby a plurality of charge pulses of two or more biphasic waveforms are alternatingly applied, this process can be performed using a single driver (as opposed to multiple drivers).

Although FIG. 10 illustrates an example where a biphasic waveform includes both a plurality of charge pulses and a plurality of discharge pulses, the present disclosure is not limited thereto. In some other examples, a biphasic waveform may include a plurality of charge pulses and a single discharge pulse. For example, the first biphasic waveform 1031 may have the plurality of first charge pulses 1091 and a single first discharge pulse instead of the plurality of first discharge pulses 1093, and the second biphasic waveform 1032 may have the plurality of first charge pulses 1094 and a single second discharge pulse instead of the plurality of second discharge pulses 1096. The single first discharge pulse and the single second discharge pulse may have parameters (e.g., amplitude and duration) that achieve charge balancing respectively with the plurality of first charge pulses 1091 and the plurality of second charge pulses 1094. The single first discharge pulse and the single second discharge pulse may be separated in time and applied by a single driver via one or more switches, similar to how the first and second discharge pulses 493 and 496 of the example of FIGS. 4 and 5 are applied at separate times. In some other examples, a passive discharge phase may be used to remove the charge applied to electrode contacts by the pluralities of first and second charge pulses 1091 and 1094, which may entail shorting together all of the electrode contacts (or at least all of the electrode contacts charged by the first and second charge pulses 1091 and 1094).

Although FIG. 10 has been described according to some examples in terms of providing unipolar stimulation, the present disclosure also encompasses examples where bipolar stimulation is instead provided.

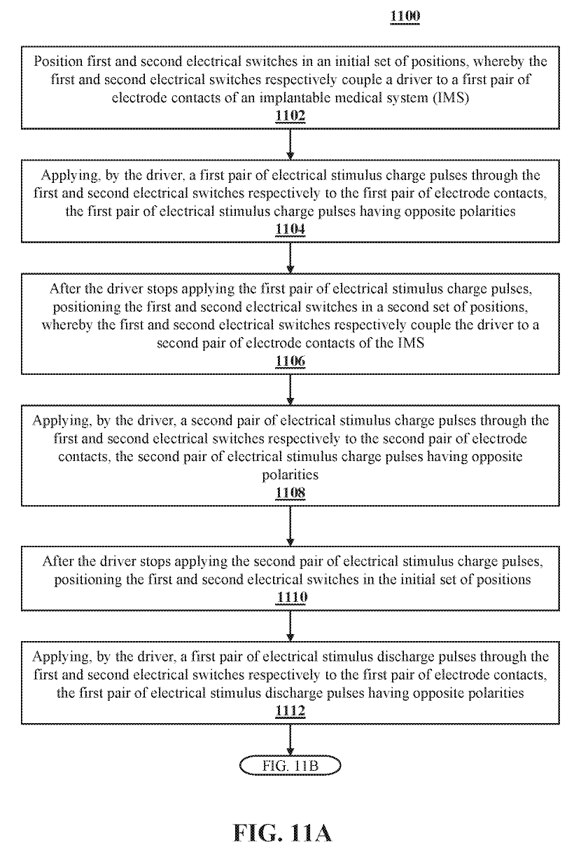

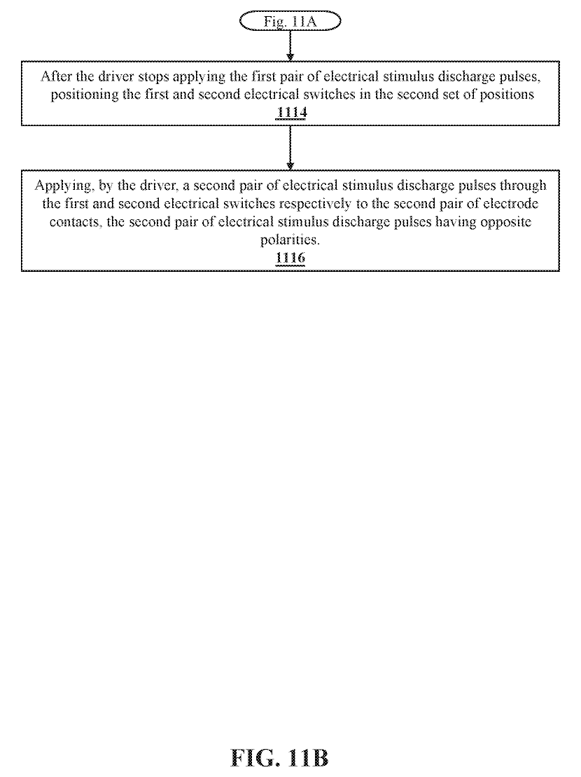

FIGS. 11A and 11B illustrate a flow chart for a process 1100 for providing stimulation to biological tissue. The process 1100 may be performed by an IMS, for example, the IMS 110 described herein with reference to FIGS. 1-3.

The process 1100 may include a first operation 1102 of positioning first and second electrical switches in an initial set of positions, whereby the first and second electrical switches respectively couple a driver to a first anode-cathode pair of electrode contacts of an IMS. For example, the first and second electrical switches may be the first and second electrical switches 316A and 316B of the IMS 110, and the first anode-cathode pair of electrode contacts may be two electrode contacts from among the first to fifth electrode contacts 351-355.

The process 1100 may include a second operation 1104 of applying, by the driver, a first pair of charge pulses through the first and second electrical switches respectively to the first pair of anode-cathode electrode contacts, wherein the first pair of charge pulses have opposite polarities. For example, the first pair of charge pulses may include the charge pulses 491 of the first and second biphasic waveforms 431 and 432 discussed with reference to FIGS. 4-5. The second operation 1104 may occur after the first operation 1102.

The process 1100 may include a third operation 1106 of, after the driver stops applying the first anode-cathode pair of charge pulses, positioning the first and second electrical switches in a second set of positions, whereby the first and second electrical switches respectively couple the driver to a second anode-cathode pair of electrode contacts of the IMS. In some examples, the second anode-cathode pair of electrode contacts may be two electrode contacts from among the first to fifth electrode contacts 351-355, and one or both contacts of the second anode-cathode pair of electrodes contacts is different from each of the first anode-cathode pair of electrode contacts.

The process 1100 may include a fourth operation 1108 of applying, by the driver, a second anode-cathode pair of charge pulses through the first and second electrical switches respectively to the second anode-cathode pair of electrode contacts, wherein the second anode-cathode pair of charge pulses have opposite polarities. For example, the second anode-cathode pair of charge pulses may include the charge pulses 494 of the third and fourth biphasic waveforms 433 and 434 described with reference to FIGS. 4-5. The fourth operation 1108 may occur after the third operation 1106.

The process 1100 may include a fifth operation 1110 of, after the driver stops applying the second pair of charge pulses, positioning the first and second electrical switches back in the initial set of positions.

The process 1100 may include a sixth operation 1112 of applying, by the driver, a first anode-cathode pair of discharge pulses through the first and second electrical switches respectively to the first anode-cathode pair of electrode contacts, wherein the first anode-cathode pair of discharge pulses have opposite polarities. For example, the discharge pulses 493 are applied to the first anode-cathode pair of electrode contacts.

The process 1100 may include a seventh operation 1114 of, after the driver stops applying the first anode-cathode pair of discharge pulses, positioning the first and second electrical switches back in the second set of positions.

The process 1100 may include an eighth operation 1116 of applying, by the driver, a second anode-cathode pair of discharge pulses through the first and second electrical switches respectively to the second anode-cathode pair of electrode contacts, the second pair of discharge pulses having opposite polarities. For example, the discharge pulses 496 are applied to the second anode-cathode pair of electrode contacts.

During the process 1100, the driver may apply the first anode-cathode pair of charge pulses, the second anode-cathode pair of charge pulses, the first anode-cathode pair of discharge pulses, and the second anode-cathode pair of discharge pulses via a time-multiplexing operation by switching the first and second electrical switches between the initial set of positions and the second set of positions.

During the process 1100, a first interphase delay period may be defined between a time at which the driver ceases to apply a first charge pulse of the first anode-cathode pair of charge pulses to a first electrode contact of the first anode-cathode pair of electrode contacts and a time at which the driver begins to apply a first discharge pulse of the first anode-cathode pair of discharge pulses to the first electrode contact. In some examples, the driver may not apply electrical stimulus to the first electrode contact during the first interphase delay period.

The driver may apply a second charge pulse of the second anode-cathode pair of charge pulses to a second electrode contact of the second anode-cathode pair of electrode contacts during the first interphase delay period. For example, during the first interphase delay period, the driver may be switched from the first electrode contact to the second electrode contact, and may apply the second charge pulse to the second electrode contact before being switched back to the first electrode contact.

In some examples, a second interphase delay period is defined between a time at which the driver ceases to apply the second charge pulse to the second electrode contact and when the driver begins to apply a second discharge pulse of the second anode-cathode pair of discharge pulses to the second electrode contact. In some examples, the driver may not apply electrical stimulus to the second electrode contact during the second interphase delay period.

The driver may apply the first discharge pulse to the first electrode contact during the second interphase delay period. For example, during the second interphase delay period, the driver may be switched from the second electrode contact to the first electrode contact, and may apply the first discharge pulse to the first electrode contact before being switched back to the second electrode contact.

Although some methods for applying electrical stimulus to biological tissue have been discussed with reference to FIGS. 11A-11B, the present disclosure is not limited thereto. Systems for providing electrical stimulus, and processes performed by such systems, have been described herein with reference to FIGS. 1-10, and the present disclosure includes all methods for providing electrical stimulus that include any combination of such processes in any suitable order.

Each feature of embodiments of the present disclosure may be combined with each other, partially or entirely, and may be technically interlocked and operated in various ways, and each embodiment may be implemented independently of each other or in conjunction with each other.

Although specific embodiments are described herein, the scope of the technology is not limited to those specific embodiments. Moreover, while different embodiments may be described separately, such embodiments may be combined with one another in implementing the technology described herein. One skilled in the art will recognize other embodiments or improvements that are within the scope and spirit of the present disclosure. Therefore, specific examples are disclosed only as illustrative embodiments. The scope of the technology is defined by the following claims and any equivalents therein.

What is claimed is:

1. A medical system, comprising:
an implantable pulse generator (IPG) configured for current steering;
a lead electrically coupled to the IPG; and
a stimulation electrode on the lead and comprising a plurality of separately chargeable electrode contacts,
wherein the IPG comprises a driver configured to selectively apply electrical stimulus to the plurality of electrode contacts, and
wherein the IPG is configured to:
cause the driver to apply first electrical stimulus to a first electrode contact of the plurality of electrode contacts; and
subsequently cause, while the first electrical stimulus is stored on the first electrode contact, the driver to apply second electrical stimulus to a second electrode contact.

2. The medical system of claim 1, wherein the IPG is configured to cause the first electrode contact to hold the first electrical stimulus during a first interphase period and to cause the driver to discharge the first electrical stimulus after the first interphase period.

3. The medical system of claim 2, wherein the IPG is configured to cause the driver to apply the second electrical stimulus to the second electrode contact during the first interphase period.

4. The medical system of claim 1, wherein the stimulation electrode is a nerve cuff electrode configured to wrap around a nerve.

5. The medical system of claim 1, wherein the second electrode contact is of the plurality of electrode contacts of the stimulation electrode.

6. The medical system of claim 1, wherein the second electrode contact is separated from the stimulation electrode.

7. The medical system of claim 6, wherein the second electrode contact is on the IPG.

8. The medical system of claim 1, wherein the IPG comprises a microcontroller circuit operatively coupled to the driver.

9. The medical system of claim 1, wherein the first and second electrical stimuli have the same polarity, and the driver is configured to differentially apply the first and second electrical stimuli with different amplitudes.

10. The medical system of claim 1, wherein the IPG is configured to cause the second electrode contact to hold the second electrical stimulus during a second interphase period, and to discharge the second electrical stimulus after the second interphase period.

11. The medical system of claim 10, wherein the IPG is configured to cause the driver to discharge the first electrical stimulus during the second interphase period.

12. The medical system of claim 10, wherein the IPG is configured to cause the first and second electrode contacts to respectively discharge the first and second electrical stimuli by a plurality of discharge pulses.

13. The medical system of claim 12, wherein the plurality of discharge pulses for discharging the first electrode contact are alternated with the plurality of discharge pulses for discharging the second electrode contact.

14. The medical system of claim 1, wherein the driver is configured to apply, while applying the first electrical stimulus to the first electrode contact, a third electrode contact with third electrical stimulus of opposite polarity to the first electrical stimulus.

15. The medical system of claim 14, wherein the first electrical stimulus is cathodic electrical stimulus, the second electrical stimulus is cathodic electrical stimulus, the third electrical stimulus is anodic electrical stimulus, the second electrode contact is of the plurality of electrode contacts of the stimulation electrode, and the third electrode contact is separated from the stimulation electrode.

16. The medical system of claim 14, wherein the first electrical stimulus is anodic electrical stimulus, the second electrical stimulus is anodic electrical stimulus, the third electrical stimulus is cathodic electrical stimulus, and the second and third electrode contacts are each of the plurality of electrode contacts of the stimulation electrode, and
wherein the driver is configured to apply, while applying the anodic electrical stimulus to the second electrode contact, additional cathodic electrical stimulus to the third electrode contact.

17. A medical system, comprising:
an implantable pulse generator (IPG);
a lead electrically coupled to the IPG; and
a stimulation electrode on the lead and comprising a plurality of separately chargeable electrode contacts,
wherein the IPG comprises:
a driver configured to apply electrical stimulus; and
a first electrical switch electrically coupled to the driver and positionable in at least a first position, whereby the first electrical switch is coupled to a first electrode contact of the plurality of electrode contacts, and a second position, whereby the first electrical switch is coupled to a second electrode contact.

18. The medical system of claim 17, wherein the stimulation electrode is a nerve cuff electrode, and the plurality of electrode contacts are configured to be arranged to at least partially circumferentially surround the nerve when the nerve cuff electrode is wrapped around the nerve.

19. The medical system of claim 17, wherein the second electrode contact is of the plurality of electrode contacts of the stimulation electrode.

20. The medical system of claim 17, wherein the second electrode contact is separated from the stimulation electrode.

21. The medical system of claim 20, wherein the second electrode contact is on the IPG.

22. The medical system of claim 17, wherein the IPG comprises a second electrical switch coupled to the driver and selectively positionable in each of multiple different positions, whereby the second electrical switch is respectively coupled to multiple different electrode contacts, the multiple different electrode contacts comprising the first and second electrode contacts, and
wherein the first electrical switch is selectively positionable in each of the multiple different positions independently of the position of the second electrical switch.

23. The medical system of claim 22, wherein the IPG is configured to cause the driver to be coupled, via the first and second electrical switches, to two different electrode contacts of the multiple different electrode contacts, and to apply cathodic electrical stimulus to one of the two different electrode contacts and corresponding anodic electrical stimulus to another one of the two different electrode contacts.

24. The medical system of claim 17, wherein the IPG further comprises a microcontroller circuit operatively coupled to the driver and to the first electrical switch.

25. The medical system of claim 24, wherein the microcontroller circuit is configured to cause the driver to apply electrical stimulus to at least some of the plurality of electrode contacts via a time-multiplexing operation of the first electrical switch, to generate a combined electric field defined at least in part by the combination of individual electric fields respectively associated with the at least some of the plurality of electrode contacts.

26. The medical system of claim 24, wherein the microcontroller circuit is configured to cause the driver to differentially charge two electrode contacts of the plurality of electrode contacts with different amplitudes of an anodic charge, or with different amplitudes of a cathodic charge, to generate a combined electric field defined at least in part by the combination of individual electric fields respectively associated with the two charged electrode contacts.

27. The medical system of claim 24, wherein the microcontroller circuit is configured to:
electrically couple, via the first electrical switch, the driver to the first electrode contact and cause the driver to apply first electrical stimulus to the first electrode contact; and
electrically couple, via the first electrical switch, the driver to the second electrode contact and cause, while the first electrical stimulus is applied to the first electrode contact, the driver to apply second electrical stimulus to the second electrode contact.

28. The medical system of claim 27, wherein the microcontroller circuit is configured to electrically couple, via the first electrical switch, the driver to a third electrode contact and cause, while the first and second electrical stimuli are respectively applied to the first and second electrode contacts, the driver to apply third electrical stimulus to the third electrode contact.

29. The medical system of claim 27, wherein the first electrical stimulus is cathodic electrical stimulus, and the microcontroller is configured to cause the driver to apply a third electrode contact with anodic electrical stimulus while the driver is applying the first electrode contact with the cathodic electrical stimulus.

30. The medical system of claim 29, wherein the third electrode contact is separated from the stimulation electrode, the second electrode contact is of the plurality of electrode contacts of the stimulation electrode, and the second electrical stimulus is cathodic electrical stimulus.

31. The medical system of claim 27, wherein the first electrical stimulus is anodic electrical stimulus, the second electrode contact is of the plurality of electrode contacts of the stimulation electrode, the second electrical stimulus is anodic electrical stimulus, and the microcontroller circuit is configured to cause the driver to:
apply cathodic electrical stimulus to a third electrode contact of the plurality of electrode contacts of the stimulation electrode while the driver is applying the first electrode contact with the anodic electrical stimulus; and
apply additional cathodic electrical stimulus to the third electrode contact while the driver is applying the first electrode contact with the anodic electrical stimulus.

32. The medical system of claim 27, wherein the microcontroller circuit is configured to:
cause the first electrode contact to receive the first electrical stimulus via a first pulse provided by the driver, to hold the first electrical stimulus during a first interphase period, and to discharge the first electrical stimulus after the first interphase period; and
cause the second electrode contact to receive the second electrical stimulus via a second pulse provided by the driver during the first interphase period, to hold the second electrical stimulus during a second interphase period, and to discharge the second electrical stimulus after the second interphase period.

33. The medical system of claim 32, wherein the microcontroller circuit is configured to:
cause the first electrode contact to discharge the first electrical stimulus via a plurality of first discharge pulses; and
cause the second electrode contact to discharge the second electrical stimulus via a plurality of second discharge pulses.

34. The medical system of claim 33, wherein the plurality of second discharge pulses alternate with the plurality of first discharge pulses.

35. The medical system of claim 27, wherein the microcontroller circuit is configured to:
cause the first electrode contact to receive the first electrical stimulus via a plurality of first charge pulses provided by the driver, to hold the first electrical stimulus during a first interphase period, and to discharge the first electrical stimulus after the first interphase period; and
cause the second electrode contact to receive the second electrical stimulus via a plurality of second charge pulses provided by the driver, to hold the second electrical stimulus during a second interphase period, and to discharge the second electrical stimulus after the second interphase period.

36. The medical system of claim 35, wherein the plurality of second charge pulses alternate with the plurality of first charge pulses.

37. The medical system of claim 36, wherein the microcontroller circuit is configured to:
cause the first electrode contact to discharge the first electrical stimulus via a plurality of first discharge pulses; and
cause the second electrode contact to discharge the second electrical stimulus via a plurality of second discharge pulses that alternate with the plurality of first discharge pulses.

38. A method for stimulating a nerve, the method comprising providing electric current steering to the nerve by:
applying, via a driver, first electrical stimulus to a first electrode contact of a stimulation electrode; and
subsequently applying, via the driver and while the first electrical stimulus is stored on the first electrode contact, second electrical stimulus to a second electrode contact of the stimulation electrode.

39. The method of claim 38, wherein the first and second electrical stimuli have the same polarity.

40. The method of claim 39, wherein the first and second electrical stimuli have different amplitudes.

41. The method of claim 39, wherein the first and second electrical stimuli are both cathodic electrical stimulus.

42. The method of claim 41, further comprising applying anodic electrical stimulus to a third electrode contact separated from the stimulation electrode.

43. The method of claim 42, wherein part of the anodic electrical stimulus is applied by the driver while the driver applies the cathodic electrical stimulus to the first electrode contact, and another part of the anodic electrical stimulus is applied to the third electrode contact while the driver applies the cathodic electrical stimulus to the second electrode contact.

44. The method of claim 42, wherein the third electrode contact is on an implantable pulse generator that is configured to apply electrical stimulus to the stimulation electrode through a lead.

45. The method of claim 39, wherein the first and second electrical stimuli are both anodic electrical stimulus, and the method further comprises applying cathodic electrical stimulus to a third electrode contact of the stimulation electrode.

46. The method of claim 45, wherein part of the cathodic electrical stimulus is applied by the driver to the third electrode contact while the driver is applying the anodic electrical stimulus to the first electrode contact, and another part of the cathodic electrical stimulus is applied by the driver to the third electrode contact while the driver is applying the anodic electrical stimulus to the second electrode contact.

47. The method of claim 38, wherein the first and second electrical stimuli have opposite polarities.

48. The method of claim 47, wherein the first and second electrical stimuli have different amplitudes.

49. The method of claim 38, further comprising applying, via the driver and while the driver applies the first electrical stimulus to the first electrode contact, third electrical stimulus to a third electrode contact, the third electrical stimulus having opposite polarity to the first electrical stimulus.

50. The method of claim 38, comprising applying, via the driver and while the first and second electrical stimuli are stored on the first and second electrode contacts, third electrical stimulus to a third electrode contact.

51. The method of claim 38, wherein the first electrical stimulus is applied to the first electrode contact by a first stimulus pulse provided by the driver, the first electrode contact holds the first electrical stimulus during a first interphase period, and the driver discharges the first electrical stimulus from the first electrode contact after the first interphase period and by one or more second stimulus pulses of opposite polarity to the first stimulus pulse, and
wherein the second electrical stimulus is applied to the second electrode contact during the first interphase period and by a third stimulus pulse.

52. The method of claim 51, wherein the driver electrically decouples from the first electrode contact during the first interphase period.

53. The method of claim 51, wherein the second electrode contact holds the second electrical stimulus during a second interphase period, and the driver discharges the second electrical stimulus from the second electrode contact after the second interphase period and by a fourth stimulus pulse of opposite polarity to the third stimulus pulse.

54. The method of claim 51, further comprising discharging the first electrical stimulus from the first electrode contact via a plurality of second stimulus pulses, and discharging the second electrical stimulus from the second electrode contact via a plurality of third stimulus pulses.

55. The method of claim 54, wherein the plurality of second stimulus pulses are alternated with the plurality of third stimulus pulses.

56. The method of claim 38, wherein the driver comprises only a single current source configured to provide current in a single direction, and
wherein the single current source is configured to be functionally bi-directional via at least one switch connected to the single current source.

57. The method of claim 38, wherein the driver comprises only a single, bi-directional current source.
